(19) United States
(12) Patent Application Publication
(10) Pub. No.: US 2026/0000435 A1
(43) Pub. Date: Jan. 1, 2026

(54) CAMERA ADAPTERS FOR SPINE RETRACTORS

(71) Applicant: Medos International Sàrl, Le Locle (CH)

(72) Inventors: Tyler Freeto, Stoughton, MA (US); Brayden Tetreault, Raynham, MA (US); Roman Lomeli, Plymouth, MA (US); Justin Fisk, Franklin, MA (US); Eric Buehlmann, Duxbury, MA (US); James Murray, Quincy, MA (US); John DiVincenzo, Weymouth, MA (US); Marissa Maciel, Bristol, RI (US)

(21) Appl. No.: 19/254,562

(22) Filed: Jun. 30, 2025

Related U.S. Application Data
(60) Provisional application No. 63/665,859, filed on Jun. 28, 2024.

(51) Int. Cl. A61B 17/70; A61B 17/56; A61B 17/68; A61B 90/00; A61F 2/44; A61F 2/46

(52) U.S. Cl. CPC A61B 17/7074; A61B 90/361; A61F 2/4455; A61F 2/4684; A61B 2017/564; A61B 2017/681; A61B 2090/3612

(57) ABSTRACT

Disclosed are surgical instruments and methods that aid in positioning a surgical visualization system, such as a camera or other imaging device, relative to an access device in a surgical procedure, including spinal procedures. The disclosed instruments and methods provide for mounting a camera or other imaging device to an access device, such as a spinal retractor blade, at various radial positions about a working channel to facilitate use of the camera or imaging device to aid in a variety of surgical steps or procedures.

### CROSS-REFERENCE TO RELATED APPLICATIONS

This application claims the benefit of U.S. Provisional Application No. 63/665,859, entitled “Camera Adapters for Spine Retractors,” filed on Jun. 28, 2024. The entire contents of this application are incorporated by reference herein.

### FIELD

This disclosure relates generally to surgical instruments and methods of use and, more particularly, to such instruments and methods that aid in positioning a surgical visualization system during surgical procedures, including spinal procedures.

### BACKGROUND

There are many instances in which it may be desirable to provide a surgeon or other user with visualization of a surgical site. While a number of surgical visualization systems have been developed, many are heavy and cumbersome to use, difficult to clean or sterilize, or have properties that render them inadequate or impossible to use for many types of procedures. In certain spine surgeries, e.g., minimally-invasive transforaminal lumbar interbody fusion (TLIF), there are systems that provide a patient-mounted access port for reaching the surgical site and a camera disposed in the access port to provide visualization during the procedure.

Other surgical approaches exist, however, and many suffer from challenges associated with visualization of the surgical site. For example, in certain spinal procedures, e.g., lateral lumbar interbody fusion (LLIF) and anterolateral interbody fusion (ATP), bladed expandable retractors are utilized to provide access to a surgical site from a lateral surface of the patient's skin to the spine. These retractors can often be mounted to an operating table or other structure to aid their positioning relative to a patient. Visualization in lateral procedures, often utilizing microscopes or loupes, can be challenging due to the long, narrow working corridor defined by the retractor blades that can often be blocked by a surgeon's instruments or hands, as well as a lack of light reaching the surgical site at the bottom or end of the working corridor.

Accordingly, there is a need for improved instruments and methods to aid in positioning a surgical visualization system, such as a camera or other imaging device, relative to an access device, such as a bladed expandable retractor.

### SUMMARY

The present disclosure addresses the above-noted challenges and generally relates to surgical instruments and methods that aid in positioning a surgical visualization system, such as a camera or other imaging device, relative to an access device in a surgical procedure, including spinal procedures. The instruments and methods disclosed herein provide ways to mount a camera or other imaging device to a generic access device, such as a spinal retractor blade, to facilitate use of the camera or imaging device in procedures that do not utilize an access port specifically configured to accommodate the camera. As a result, a wide variety of surgical procedures can benefit from improved visualization, including all spine procedures, such as transforaminal lumbar interbody fusion (TLIF), lateral lumbar interbody fusion (LLIF), anterolateral interbody fusion (ATP), anterior lumbar interbody fusion (ALIF), anterior cervical discectomy and fusion (ACDF), and posterior cervical approaches. Such benefits are possible when using minimally-invasive (MIS) and mini-open approaches to these procedures, among others.

In one aspect, a surgical method can include inserting a surgical access device into a patient using a lateral approach, mounting a visualization system to a distal portion of the access device at a first position about a perimeter of a working channel defined by the access device, and coupling the surgical access device to a vertebra of the patient's spine using the visualization system to assess coupling progress. The method can also include mounting the visualization system at a second position about the perimeter of the working channel of the access device, dissecting tissue to access the patient's spine using the visualization system at the second position to assess dissection progress, and preparing the patient's spine for vertebral body fusion. The method can further include inserting the visualization system into space between adjacent vertebrae to observe results of preparing the patient's spine for vertebral body fusion.

Any of a variety of alternative or additional features can be included and are considered within the scope of the present disclosure. For example, in some embodiments, the method can further include forming an incision in a patient's skin.

In some embodiments, the method can further include adjusting a size of the working channel of the surgical access device after insertion into the patient.

In certain embodiments, inserting the surgical access device into the patient can be performed while the patient is in a lateral decubitus position.

In some embodiments, inserting the surgical access device into the patient can be performed while the patient is in a prone position.

In certain embodiments, dissecting tissue to access the patient's spine can include approaching the spine anterior to the psoas muscle.

In some embodiments, dissecting tissue to access the patient's spine can include dissecting through the psoas muscle.

In certain embodiments, the method can further include mounting the surgical access device to a surgical table upon which the patient is disposed.

In some embodiments, the method can further include mounting an additional retractor blade to the surgical access device after the surgical access device is inserted into the patient.

In certain embodiments, the first position for mounting the visualization system can be oriented to view the patient's spine from a cranial or caudal direction.

In some embodiments, the second position for mounting the visualization system can be oriented to view the patient's spine from an anterior or posterior direction.

In certain embodiments, the method can further include returning the visualization system to the second position after inserting the visualization system into the space between adjacent vertebrae.

In some embodiments, the method can further include inserting one or more implant trialing instruments into the space between adjacent vertebrae using the visualization system to assess required implant size.

In certain embodiments, the method can further include inserting bone graft material using the visualization system to assess insertion progress.

In some embodiments, the method can further include inserting an intervertebral implant using the visualization system to assess implant positioning.

In certain embodiments, the method can further include separating the surgical access device from the patient's spine using the visualization system to assess separation progress.

In some embodiments, the method can further include removing the surgical access device from the patient while using the visualization system to monitor muscle and/or tissue during removal.

In certain embodiments, the visualization system can include a camera sensor disposed at a distal end thereof such that mounting the visualization system to the distal portion of the access device includes inserting the camera senor into the patient.

In another aspect, a surgical method can include inserting a distal portion of an access device into a patient through an incision, the access device defining a working channel therethrough, and mounting a camera system into the working channel of the access device at a first position around a perimeter of the working channel. The method can further include coupling the access device to a support arm, and docking the access device to an anatomical structure of the patient at a surgical site, wherein the camera system is configured to view the anatomical structure in the first position. The method can also include repositioning the camera to a second position around the perimeter of the working channel, and performing one or more surgical procedures at the surgical site.

In some embodiments, a plurality of camera systems can be mounted at a plurality of positions around the perimeter of the access device.

In certain embodiments, the working channel can extend medially from a lateral skin surface towards a spine of the patient.

In some embodiments, the working channel can extend through a psoas muscle. In certain embodiments, the patient can be positioned in a lateral decubitus position and the working channel can extend perpendicular to a ground surface.

In certain embodiments, the patient can be positioned in a prone position and the working channel can extend parallel to a ground surface.

In some embodiments, the surgical access device can include a retractor body including first arm, a second arm opposed to the first arm, and a third arm; and first, second, and third retractor blades respectively coupled to each arm and defining the working channel longitudinally therebetween. In certain embodiments, the method can further include moving each retractor blade relative the other retractor blades to retract soft tissue. In some embodiments, the camera system can be mounted to the first retractor blade and can view the surgical site from the caudal direction in the first position. In certain embodiments, the camera system can be mounted to the second retractor blade and can view the surgical site from the cranial direction in the second position. In some embodiments, the camera system can be mounted to the third retractor blade and can view the surgical site from the posterior direction in the second position.

In certain embodiments, the camera system can include an adaptor configured to slidably couple the camera system to a retractor blade of the access device. In some embodiments, the method can further include adjusting the position of the camera system along a length of the retractor blade, wherein the retractor blade defines a portion of the working channel. In certain embodiments, the camera system can be positioned proximal to a distal end of the retractor blade and configured to visualize docking of the distal end of the access device.

In some embodiments, the surgical access device can include a handheld retractor assembly configured to be selectively coupled to the retractor body. The handheld retractor assembly can include a fourth retractor blade and a blade holder coupled to a proximal end of the fourth retractor blade. In certain embodiments, the method can further include mounting the camera system to a distal end of the handheld retractor assembly, and inserting a distal end of the removable retractor blade into the incision through soft tissue towards the surgical site. The method can also include retracting soft tissue with the handheld retractor assembly while visualizing the soft tissue through the camera system, and coupling the handheld retractor assembly to the access device. In some embodiments, the camera system can view the surgical site from an anterior direction when mounted to the handheld retractor assembly. In certain embodiments, the working channel can extend anterior to a psoas muscle. In some embodiments, the handheld retractor assembly can be coupled to the access system via a connector arm coupled to a proximal end of the retractor body and the blade holder.

Any of the features or variations described herein can be applied to any particular aspect or embodiment of the present disclosure in a number of different combinations. The absence of explicit recitation of any particular combination is due solely to avoiding unnecessary length or repetition.

### BRIEF DESCRIPTION OF THE DRAWINGS

The aspects and embodiments of the present disclosure can be more fully understood from the following detailed description taken in conjunction with the accompanying drawings, in which:

FIG. 1 is perspective view of an embodiment of a surgical system that provides a patient-mounted access port for reaching a surgical site;

FIG. 2 is a perspective view an embodiment of a camera system that can be used to provide visualization of a surgical site;

FIG. 3 is a perspective view of a camera disposed within a surgical access device;

FIG. 4 is a side cross-sectional view of the camera of FIG. 2;

FIG. 5 is a perspective view of an embodiment of a three-blade expanding retractor;

FIG. 6 is a perspective view of another embodiment of a three-blade expanding retractor;

FIG. 7 is a perspective view of an embodiment of a patient-mounted bladed retractor;

FIG. 8 is a perspective view of an embodiment of a retractor system including the retractor of FIG. 5;

FIG. 9 is a perspective view of a camera and camera adaptor used in the disclosed surgical retractor systems;

FIG. 10A is a perspective view of the camera adaptor of FIG. 9;

FIG. 10B is an alternative perspective view of the camera adaptor of FIG. 10A;

FIG. 10C is a top view of the camera adaptor of FIG. 10A;

FIG. 10D is a bottom view of the camera adaptor of FIG. 10A;

FIG. 10E is a side longitudinal view of the camera adaptor of FIG. 10A;

FIG. 10F is a side end view of the camera adaptor of FIG. 10A;

FIG. 11 is a perspective longitudinal cross-sectional view the camera adaptor of FIG. 10A taken along the line A-A in FIG. 10F;

FIG. 12 is a side longitudinal cross-sectional view of the camera and camera adaptor of FIG. 9 taken along the line A-A in FIG. 9;

FIG. 13 is a perspective view of a camera coupled to a caudal blade of a retractor device;

FIG. 14 is a perspective view of a camera coupled to a posterior blade of a retractor device;

FIG. 15 is a perspective view of a blade holder of the retractor system of FIG. 8;

FIG. 16 is a perspective view of the blade holder of FIG. 15 coupled to a retractor blade;

FIG. 17 is a perspective view of the blade holder and retractor blade of FIG. 16 coupled to a camera;

FIG. 18 is a perspective view of a retractor system including the blade holder, retractor blade, and camera of FIG. 17;

FIG. 19A is a perspective view of another embodiment of a blade holder;

FIG. 19B is an alternative perspective view of the blade holder of FIG. 19A;

FIG. 19C is a first end view of the blade holder of FIG. 19A;

FIG. 19D is a side view of the blade holder of FIG. 19A;

FIG. 19E is a second end view of the blade holder of FIG. 19A;

FIG. 19F is a top view of the blade holder of FIG. 19A;

FIG. 19G is a bottom view of the blade holder of FIG. 19A;

FIG. 20A is an exploded view of the blade holder of FIG. 19A;

FIG. 20B is an alternative exploded view of the blade holder of FIG. 19A;

FIG. 21 is a longitudinal cross-sectional view of the blade holder of FIG. 19A taken along the line A-A in FIG. 19F;

FIG. 22 is a lateral cross-sectional view of the blade holder of FIG. 19A taken along the line B-B in FIG. 19F;

FIG. 23 is a perspective view of a connector arm adapter;

FIG. 24A is a perspective view of another embodiment of a connector arm adapter;

FIG. 24B is an alternative perspective view of the connector arm adapter of FIG. 24A;

FIG. 24C is a bottom view of the connector arm adapter of FIG. 24A;

FIG. 24D is a top view of the connector arm adapter of FIG. 24A;

FIG. 24E is a first end view of the connector arm adapter of FIG. 24A;

FIG. 24F is a second end view of the connector arm adapter of FIG. 24A;

FIG. 24G is a side view of the connector arm adapter of FIG. 24A;

FIG. 25 is a longitudinal cross-sectional view of the connector arm adapter of FIG. 24A taken along the line A-A in FIG. 24D;

FIG. 26A is an exploded view of the connector arm adapter of FIG. 24A;

FIG. 26B is an alternative exploded view of the connector arm adapter of FIG. 24A;

FIG. 27 is an anterior view of a retractor system providing a lateral working corridor to a surgical site in a patient in the decubitus position;

FIG. 28 is a posterior view of the retractor system of FIG. 8 coupled to a support arm;

FIG. 29 is a detail view of the retractor of FIG. 28 and a probe passed through the working corridor;

FIG. 30 shows a probe creating an entry channel through soft tissue, including a psoas muscle, to a vertebra; and

FIG. 31 shows an alternative entry channel anterior to a psoas muscle and to a vertebra.

### DETAILED DESCRIPTION

Certain example embodiments will now be described to provide an overall understanding of the principles of the structure, function, manufacture, and use of the devices, systems, and methods disclosed herein. One or more examples of these embodiments are illustrated in the accompanying drawings. The devices, systems, and methods specifically described herein and illustrated in the accompanying drawings are non-limiting embodiments. The features illustrated or described in connection with one embodiment may be combined with the features of other embodiments. Such modifications and variations are intended to be included within the scope of the present disclosure.

As noted above, the present disclosure generally relates to surgical instruments and methods that aid in positioning a surgical visualization system, such as a camera or other imaging device, relative to an access device in a surgical procedure, including spinal procedures. In particular, the present disclosure provides various adapters to facilitate positioning a camera relative to a spine retractor to allow the camera to provide improved visualization of a surgical site disposed at the end of a working corridor defined by the retractor. The instruments and methods disclosed herein can have a wide variety of applications in aiding positioning of a visualization system relative to a surgical access device, including any of a variety of approaches utilized for spine surgery.

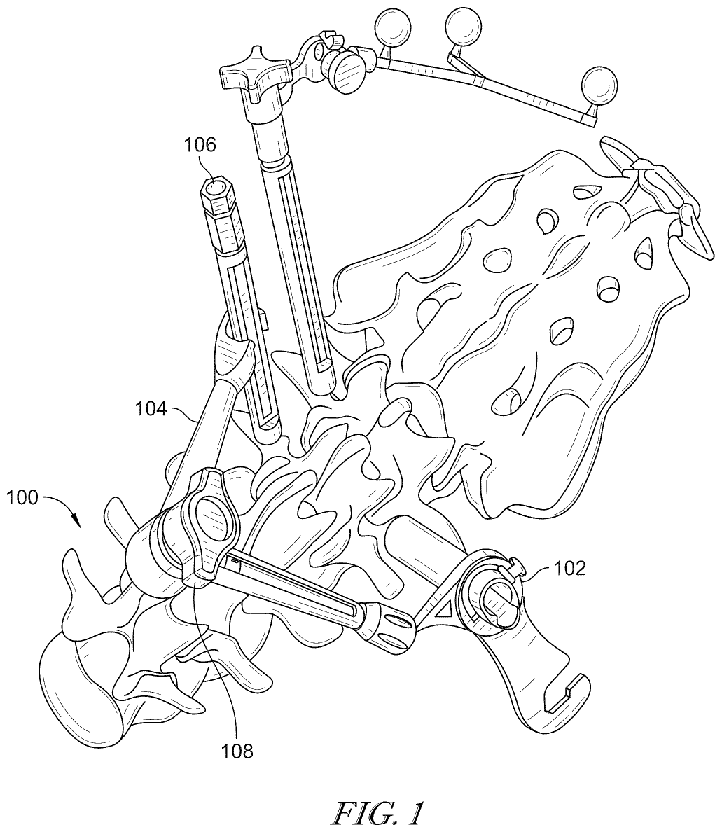

FIGS. 1-4 illustrate one embodiment of a surgical system 100 for minimally-invasive transforaminal lumbar interbody fusion (TLIF) that provides a patient-mounted access port 102 for reaching a surgical site and a camera 202 that can be disposed in the access port to provide visualization during the procedure. In general, the system 100 can include any one or more of an access device 102, a pedicle post or other anchor 106, a connector 104, and a camera 202.

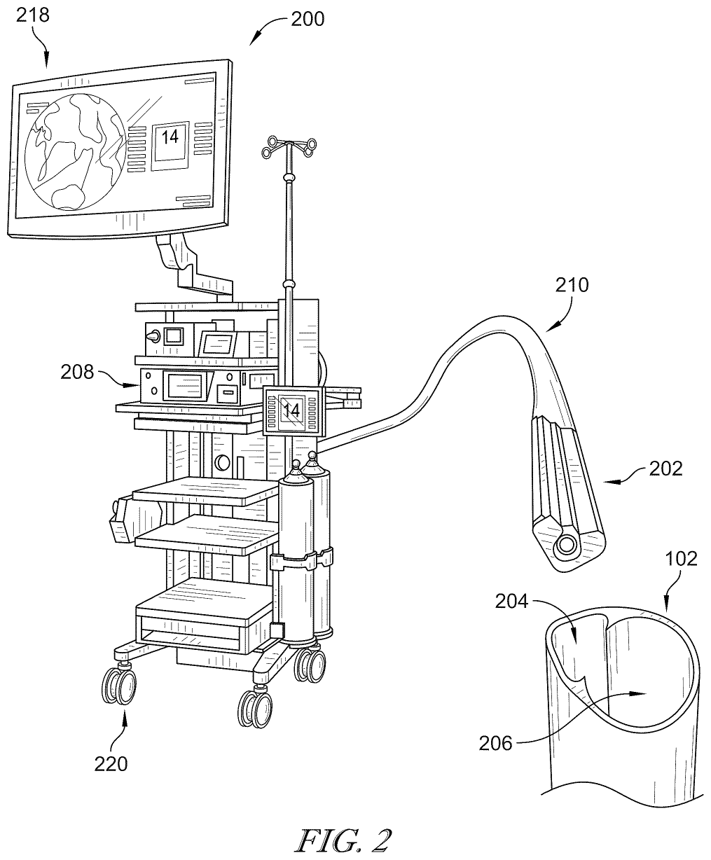

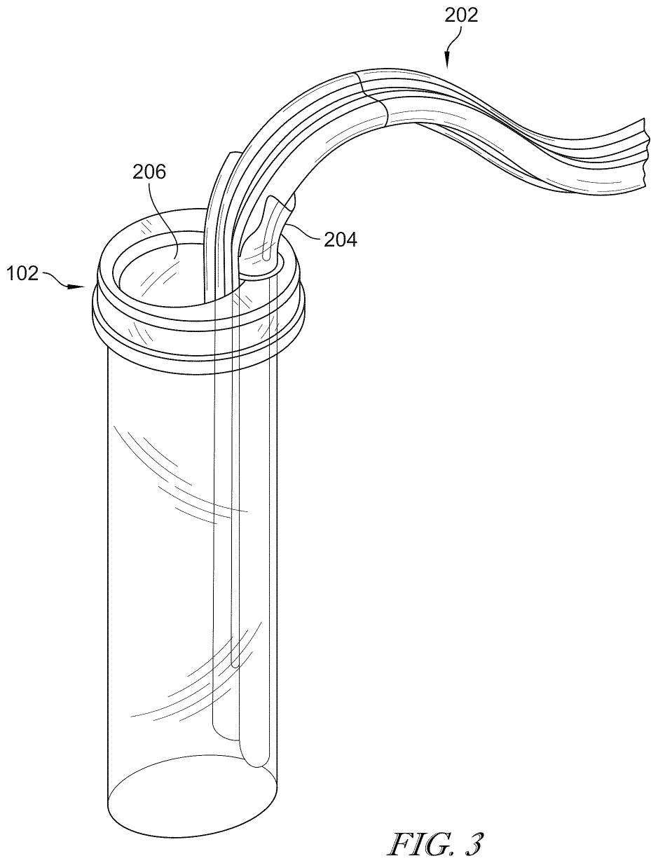

FIG. 2 illustrates one embodiment of a camera system 200 that can be used to provide visualization of a surgical site. The system 200 can include a camera or other visualization instrument 202 that can be configured to pass through the access device to reach and visualize a surgical site. For example, the camera 202 can be configured to pass through a visualization channel 204 of the access device 102 and be positioned within the channel at any of a variety of positions along the length of the access device, as shown in FIG. 3. From such a position, the camera 202 can view a working channel 206 of the access device 102 and/or a surgical site distal to a distal end of the access device. The camera 202 can be coupled to a controller 208 via one or more cables 210 in some embodiments or, in other embodiments, can communicate with a controller or other processor via wireless communication. The controller 208 can include a digital data processor, one or more storage memories, one or more inputs and outputs, and other components of electronic controllers or computing devices. The controller 208 can include one or more user interfaces for controlling the camera 202 or can be coupled to one or more input devices that can be used to control the camera 202 and/or controller 208. The controller 208 and/or camera 202 can also be coupled to one or more displays 218 that can be configured to present a variety of data to a user, including the view of a working channel and/or surgical site provided by the camera 202. The various components of the system 200 can be integrated into a mobile cart 220 as shown, or can be disposed separately about a surgical operating environment.

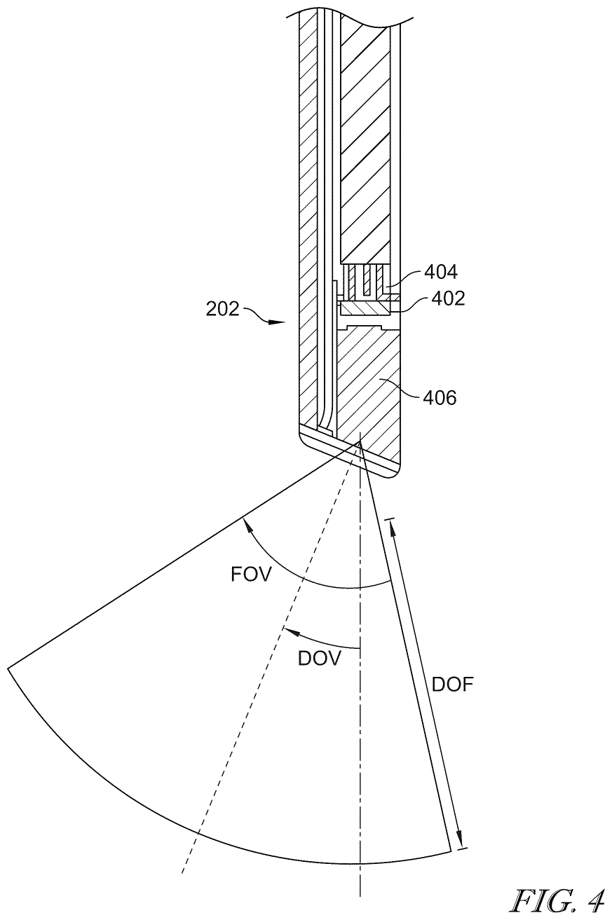

FIG. 4 illustrates a side cross-sectional view of the camera 202. The camera 202 can include an image capture sensor 402, such as a charge-coupled device (CCD) or complementary metal-oxide-semiconductor (CMOS) sensor, as well as an associated lighting device 404, such as a fiber optic that delivers light from an external source or one or more light emitting diodes (LEDs) or other light generating devices that can be integrated into the camera 202. The camera 202 can also include a lens assembly 406 that can include one or more lenses to help focus the view of the sensor 402 in a desired manner. The camera 202 can have a field-of-view (FOV), a direction of view (DOV), and a depth of field (DOF). In some embodiments, the FOV can be in the range of about 60 to about 70 degrees. In some embodiments, the DOV can be in the range of about 15 to 30 degrees. In some embodiments, the DOV can be in the range of about 20 to 25 degrees. In some embodiments, the DOV can be about 22.5 degrees. In some embodiments, the DOF can be in the range of about 7 mm to about 40 mm. Further details of example cameras can be found in US 2018/0214016, entitled “Surgical Visualization Systems and Related Methods,” the entire contents of which are incorporated by reference herein.

The system 100 provides a patient-mounted port and camera, allowing for direct visualization of the lumbar spine during a transforaminal lumbar interbody fusion (TLIF) surgical procedure. The camera 202 allows a surgeon to directly view important anatomical structures, such as nerve roots, from a perspective at the surgical site from the bottom of the minimally-invasive portal 102, thereby allowing for a smaller working channel and a view that is unobstructed by instruments or the surgeon's hands. This is preferable in comparison to more traditional methods of visualization, such as using a microscope or loupes.

Additional details regarding the system 100 and its various components, including the connector arm 104 and camera 202, can be found in the following US patents: U.S. Pat. No. 10,682,130 to White et al., U.S. Pat. No. 10,869,659 to Thommen et al., U.S. Pat. No. 10,987,129 to Thommen et al., U.S. Pat. No. 11,000,312 to Thommen et al., U.S. Pat. No. 11,331,090 to Thommen et al., U.S. Pat. No. 11,344,190 to Thommen et al., U.S. Pat. No. 11,439,380 to Thommen et al., U.S. Pat. No. 11,559,328 to Richter et al., and U.S. Pat. No. 11,771,517 to Lomeli et al. The entire contents of each of these patents are incorporated by reference herein.

The above-described surgical visualization systems and methods can, however, offer benefits to other surgical procedures as well. For example, any other spine surgery approach is likely to benefit from improved visualization. As a more particular example, lateral lumbar interbody fusion (LLIF) and anterolateral interbody fusion (ATP) procedures utilize bladed expandable retractors, with goals of reducing the amount of and time under retraction (i.e., limiting stress on muscles and nerves in the psoas being retracted). Visualization using traditional methods (e.g., microscope, loupes, etc.) can be challenging in these procedures due to the long, narrow working corridor (e.g., often longer than the portal 102 involved in TLIF procedures) that is often blocked by the surgeon's instruments or hands, as well as a lack of light reaching the surgical sight at the bottom of the access corridor. A camera disposed at the bottom of the access corridor created by the bladed retractor could allow for visual confirmation of procedure steps, such as discectomy and endplate preparation, and potentially reduce time and fluoroscopy shots required during discectomy, as well as trial and cage insertion, by allowing instrument depth markings to be read in-situ. While minimally-invasive (MIS) LLIF and ATP procedures could benefit from improved visualization, the benefits of the camera 202 could extend to most, if not all, other spine procedures as well, such as anterior lumbar interbody fusion (ALIF), anterior cervical discectomy and fusion (ACDF), posterior cervical approaches, and mini-open TLIF approaches.

The system 100 includes instrumentation to facilitate a TLIF or similar procedure that utilizes components configured to be used in a generally posterior approach to the spine. To expand the use of the camera 202 to other spine approaches, instruments and methods to mount the camera 202 to a generic retractor blade are disclosed herein. The new instruments and methods disclosed herein can allow the camera 202 and associated visualization system of FIGS. 2 and 4 to be used in other procedures and approaches, such as the LLIF and ATP approaches, among others noted herein. The disclosed instruments and methods generally fall into three categories: (1) mounting to an expanding blade retractor, (2) mounting to a generic table-mount arm, or (3) mounting to a hand-held retractor.

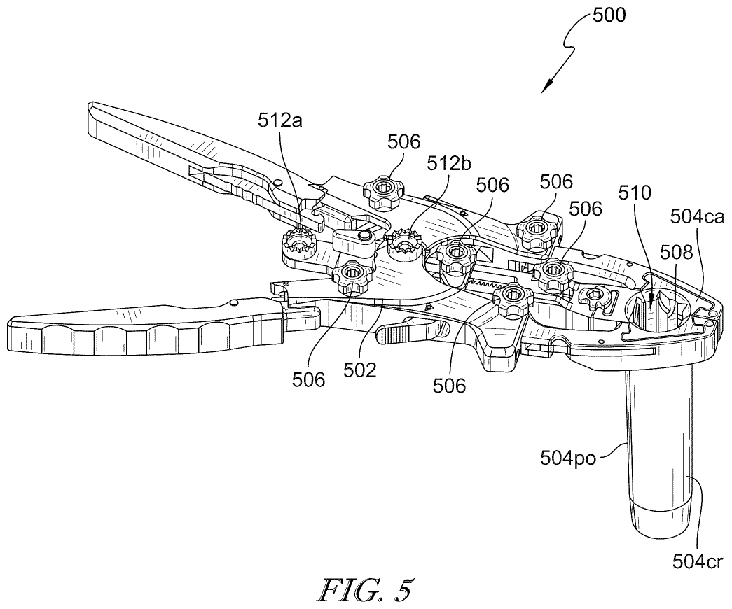

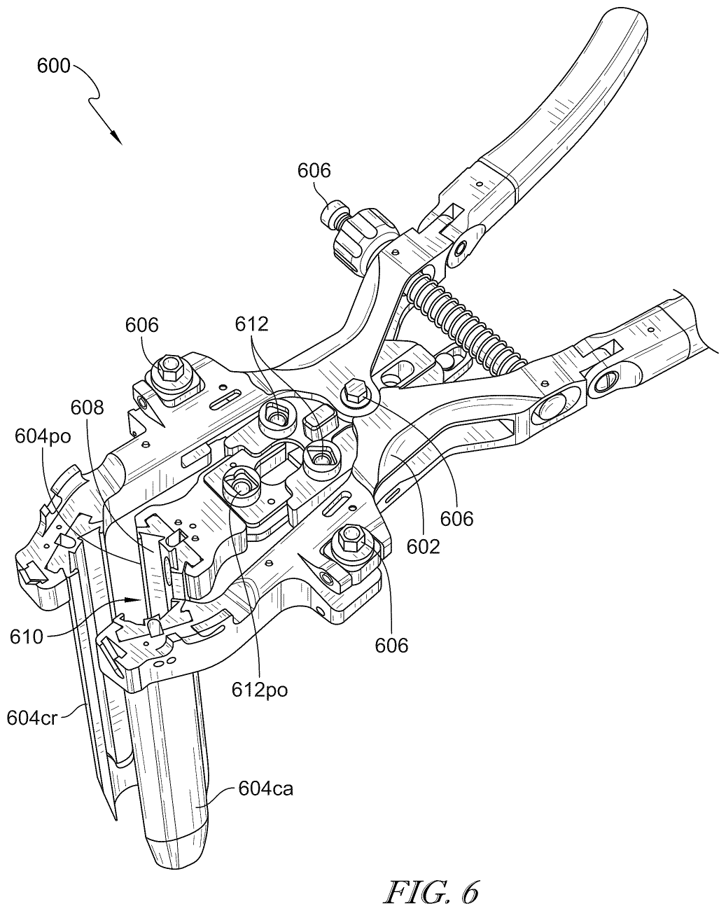

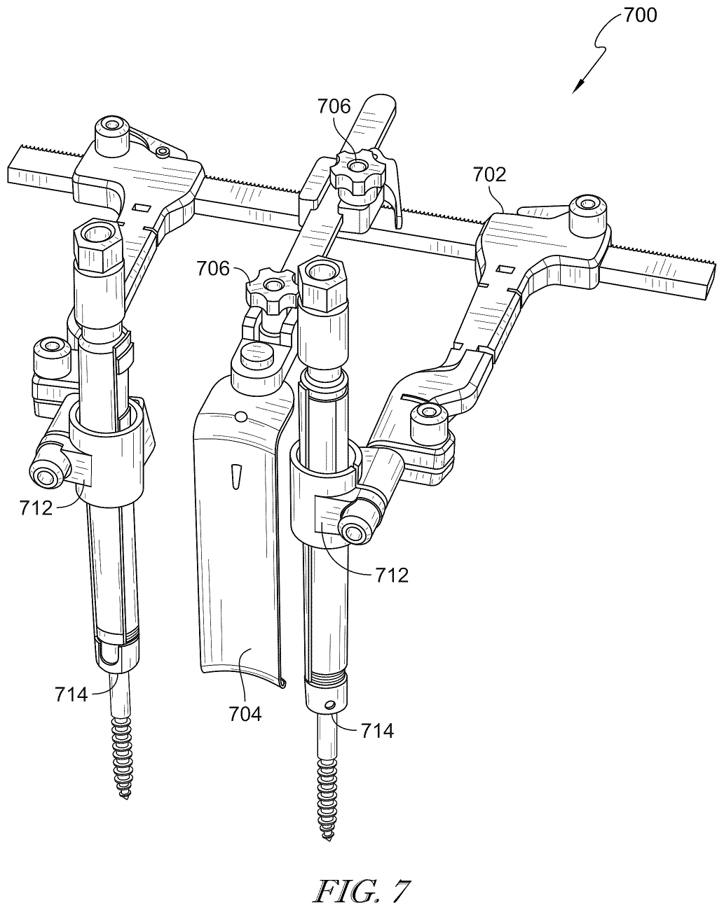

FIGS. 5-7 illustrate example expanding blade retractors that can be utilized in connection with the instruments and methods disclosed herein. FIG. 5, for example, illustrates a three-blade retractor 500 available under the trade name Phantom XL3™ by TeDan Surgical Innovations, Inc. FIG. 6 illustrates another three-blade retractor 600 available under the trade name Insight® by DePuy Synthes Spine. FIG. 7 illustrates still another example bladed retractor 700 available under the trade name Viper Prime™ by DePuy Synthes Spine. Each of these example retractors utilizes one or more retractor blades that can be positioned relative to a retractor body or frame to define a working channel or corridor for accessing a surgical site within a patient's body. In some embodiments, a surgeon can manually hold a retractor blade to manipulate tissue and aid in accessing a surgical site. The retractor blade(s) and/or frame can be mounted to an operating table, implanted screw, or other structure as appropriate based on the surgical procedure and approach being utilized. Mounting the retractor in this fashion can maintain the position of the one or more retractor blades relative to the anchoring structure, the patient's body, and the surgical site.

Returning to the example retractor 500 of FIG. 5, a retractor body 502 can couple with one or more retractor blades 504 designed to retract tissue for access to the spine (e.g., the lumbar spine in a lateral approach). In the illustrated embodiment, three retractor blades 504 are shown: a cranial retractor blade 504cr, a caudal retractor blade 504ca, and a posterior retractor blade 504po. In one example embodiment, blades between about 90 mm and about 180 mm in length can be used with the retractor, with the cranial and caudal blades 504cr, 504ca being identical to each other and the posterior blade 504po being narrower with a different attachment feature to the retractor body. Adjustment knobs 506 on the retractor body can allow for all three blades to be manipulated independently, allowing for retraction, toeing, etc. All three blades can contain a T-slot channel 508 that runs the majority of the length of the blade on a surface facing the working corridor 510 defined therebetween. The T-slot channels 508 can be used for accessories to the retractor, such as a disc shim, light source, etc. Further, the retractor body 502 can include one or more mounting points 512 that can be used to mount the retractor 500 to an arm or other structure that can, in turn, be mounted to an operating table or some other anchoring structure in order to maintain the position of the retractor relative to a patient. In the illustrated embodiment, the retractor body 502 includes a plurality of such mounting points 512a, 512b, with one mounting point 512a being fixed relative to the posterior retractor blade 504po and the other mounting point 512b being fixed relative to the retractor body 502.

The example retractor 600 of FIG. 6 similarly includes a retractor body 602, three retractor blades 604cr, 604ca, and 604po, a plurality of adjustment knobs or features 606 for adjusting the position of each blade, slotted channels 608 extending along a length of each blade, a working corridor 610 defined by the blades, and one or more mounting points 612 for coupling the retractor to an operating table or other anchoring structure. In the illustrated embodiment, three such mounting points are shown with one 612po being fixed relative to the posterior retractor blade 604po and the others being fixed relative to the retractor body 602.

The example retractor 700 of FIG. 7 similarly includes a frame structure 702, one or more retractor blades 704, one or more adjustment knobs or features 706 to facilitate adjustment of a position of the one or more blades, and one or more mounting features 712. In the illustrated embodiment, the mounting features 712 facilitate coupling the retractor 700 to implanted pedicle screws 714, e.g., in a case where a more posterior approach is being utilized. The one or more retractor blades can include any of the features described above, e.g., slotted channels extending along their length, etc.

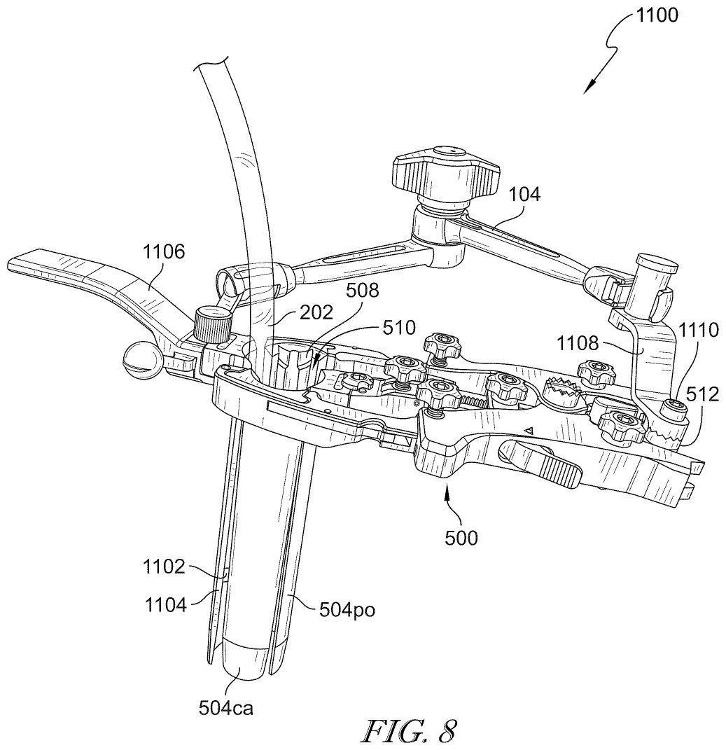

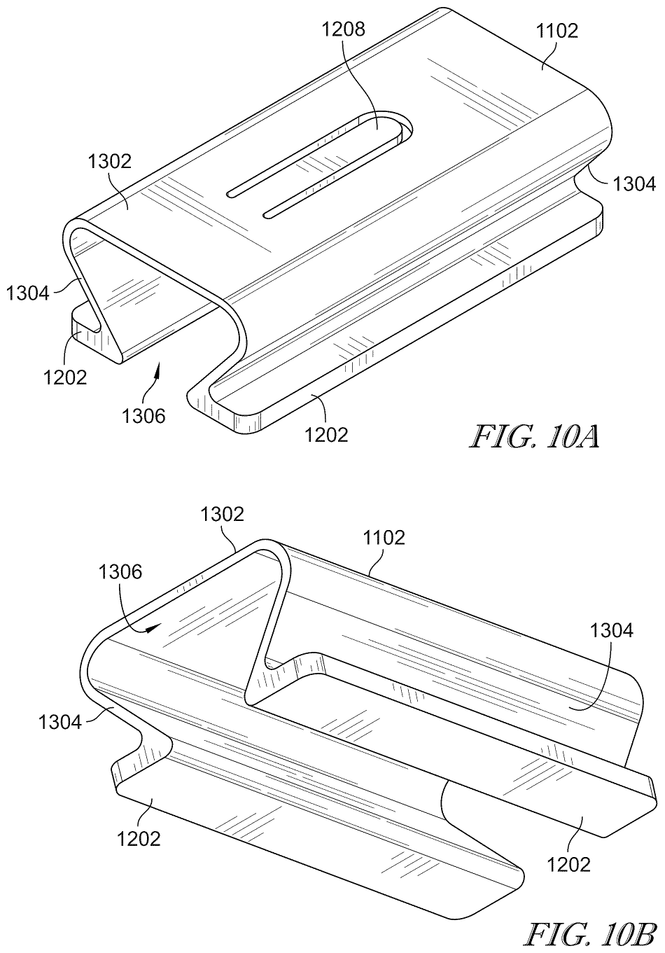

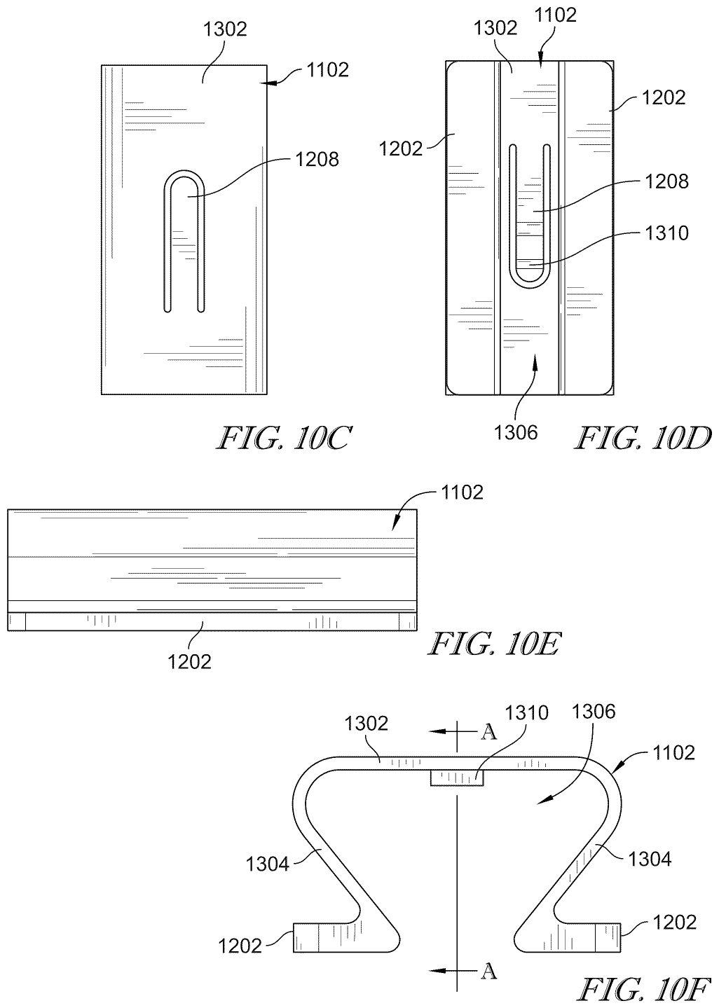

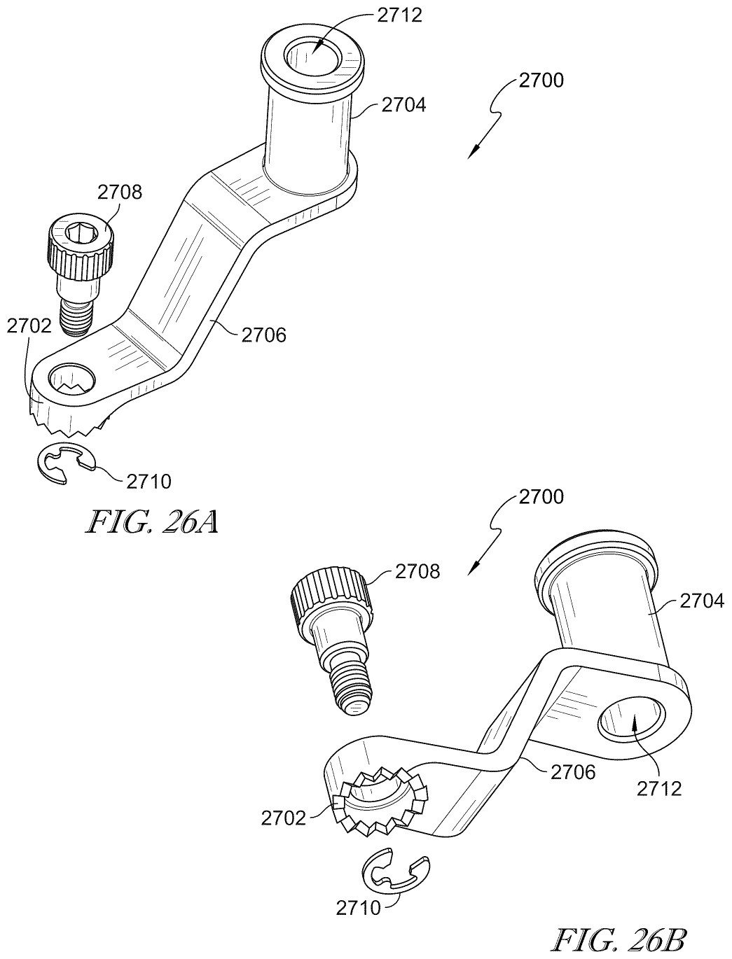

FIGS. 8-26B illustrate various example embodiments of components that can allow the above-described camera 202 or other imaging device to mount to retractor blades, e.g., by utilizing the slotted channels extending along the length of one or more retractor blades. This can allow the camera view to be adjusted by sliding the camera along the slotted channel and/or by adjusting the position and angulation of the retractor blade relative to the retractor body or frame. Also disclosed are various components to allow the positioning of a retractor blade and/or camera coupled thereto relative to a retractor frame. In some embodiments, the disclosed adapter components can facilitate positioning of a retractor blade and/or camera coupled thereto using the same kind of connector arm 104 shown in FIG. 1 and utilized (in the example embodiment of FIG. 1) to couple with an implanted pedicle screw or other bone anchor 106.

FIG. 8 illustrates a complete system 1100 including several of the adapter components disclosed herein. The system 1100 includes the example retractor 500, as well as the camera 202 coupled to an adapter 1102 that allows the camera to be positioned along a length of a fourth retractor blade 1104 (often arranged as an anteriorly-positioned retractor blade). The fourth retractor blade 1104 is illustrated coupled to a blade holder 1106 that can facilitate free-hand use of the retractor blade 1104. In addition, the blade holder 1106 can facilitate fixation of the fourth blade 1104 relative to the retractor 500 via the connector arm 104. In particular, a connector arm adapter 1108 can be coupled to the retractor body 500 and include a feature that can couple with the connector arm 104. The opposite end of the connector arm 104 can couple with the blade holder 1106. As a result, a position of the fourth retractor blade 1104 and camera 202 coupled thereto (via adapter 1102) can be adjusted and selectively fixed using the connector arm 104.

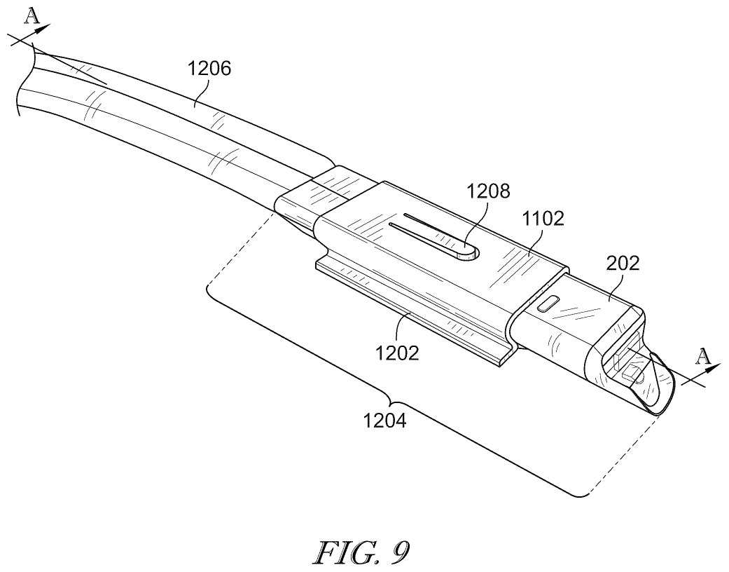

FIGS. 9-14 illustrate the camera adapter 1102 in greater detail. The camera adapter 1102 is configured to wrap around the housing of the camera 202 and provides male legs 1202 that extend from the camera housing. The camera adapter 1102 and camera 202 can have an interference fit. The legs 1202 can be received within recesses formed at the lateral edges of the T-shaped or other slots 508 extending along the length of a retractor blade. This allows the camera 202 to engage with the accessory slot found in the blades of the retractor. Additionally, the length of the slot 508 extending from the proximal most end 504p of the retractor blade along the length thereof to a distal end 504d can allow the camera to be inserted into the slot 508 from the proximal-most end 504p when the retractor is positioned in a patient. Further, the adapter 1102 and slot 508 interface can allow the position of the camera 202 to be adjusted along the length of the slot 508 from the proximal most end 504p while the retractor is positioned within a patient. The camera 202 can sit in the groove formed in the blade, allowing it to be as low-profile to the blade as possible and minimally interfere with the working channel of the retractor. As explained in more detail below, a leaf spring formed on the camera housing can provide drag against the blade itself though, in some embodiments, the legs 1202 of the camera adapter 1102 can be configured to form an interference fit with the slot 508 of the retractor.

The camera adapter 1102 can generally take the form of an open or somewhat c-shaped clip with a bridge portion 1302 extending between opposed arms 1304 that terminate in the legs 1202. The bridge portion 1302 and opposed arms 1304 can define a cavity 1306 configured to receive the camera 202, i.e., the cavity can have a cross-sectional shape that generally matches the outer shape of the body or housing of the camera (e.g., the rigid housing 1204 of the camera 202 disposed at a distal end of the flexible camera cable 1206). The legs 1202 can extend laterally outward from the ends of the opposed arms 1304 to define flat, flange-like protrusions that can extend into the lateral recesses of the accessory channels 508 formed along the retractor blade 504.

The camera adapter 1102 can include a spring finger 1208 that can provide friction and drag on the camera housing 1204 to keep the assembly together. In the illustrated embodiment, the finger 1208 is formed by a U-shaped cutout of material in the bridge portion 1302 of the camera adapter 1102. In such an embodiment, the finger 1208 can be coupled to the remainder of the bridge portion 1302 via a living hinge configuration. In other embodiments, however, the finger 1208 can be coupled to the bridge portion 1302 in another manner, e.g., by welding, adhering, or otherwise mechanically or chemically fixing discrete components. In some embodiments, the spring finger 1208 can be positioned along another portion of the camera adapter (e.g., along one of the opposed arms 1304) and, in some embodiments, multiple such features can be included (e.g., along each of the opposed arms or using multiple such features disposed along the bridge portion 1302).

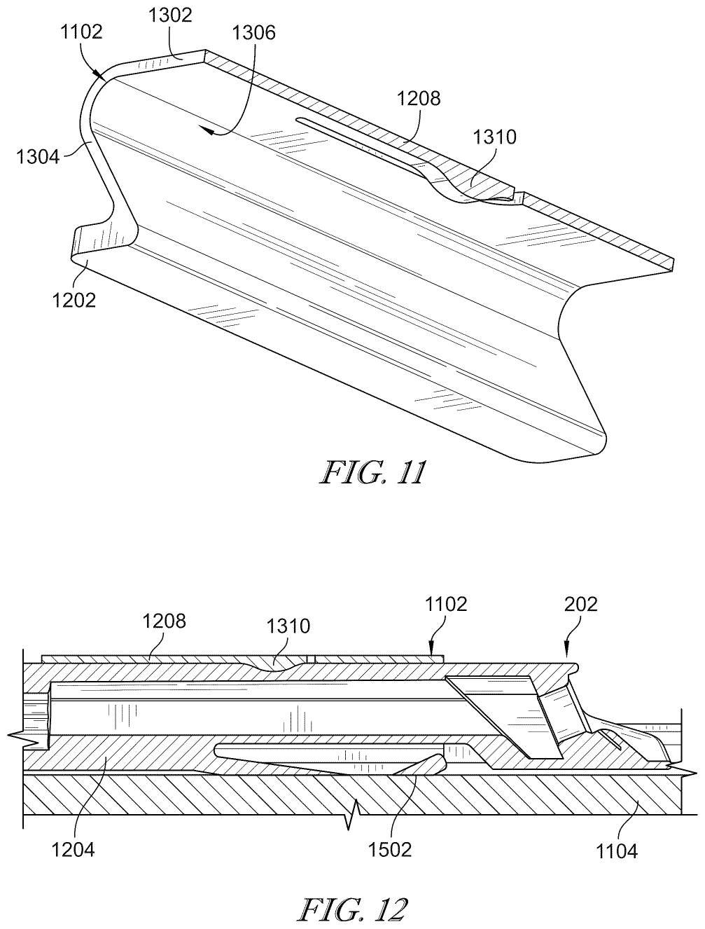

In the illustrated embodiment, the terminal free end of the spring finger 1208 can include a protrusion 1310 that extends into the cavity 1306. When the camera 202 is inserted into the cavity 1306, it can contact the protrusion 1310 and deflect the spring finger 1208 away from the cavity. The reactive force of the spring finger 1208 can provide friction to hold the camera adapter 1102 in place relative to the camera 202. Once inserted into the accessory or T-slot on the retractor blade, an exposed spring finger 1502 (see FIG. 12) on the camera housing 1204 can provide friction against the retractor blade, thereby allowing the camera and adapter assembly to move together vertically in the slot and be positioned as desired in-situ by a surgeon or other user.

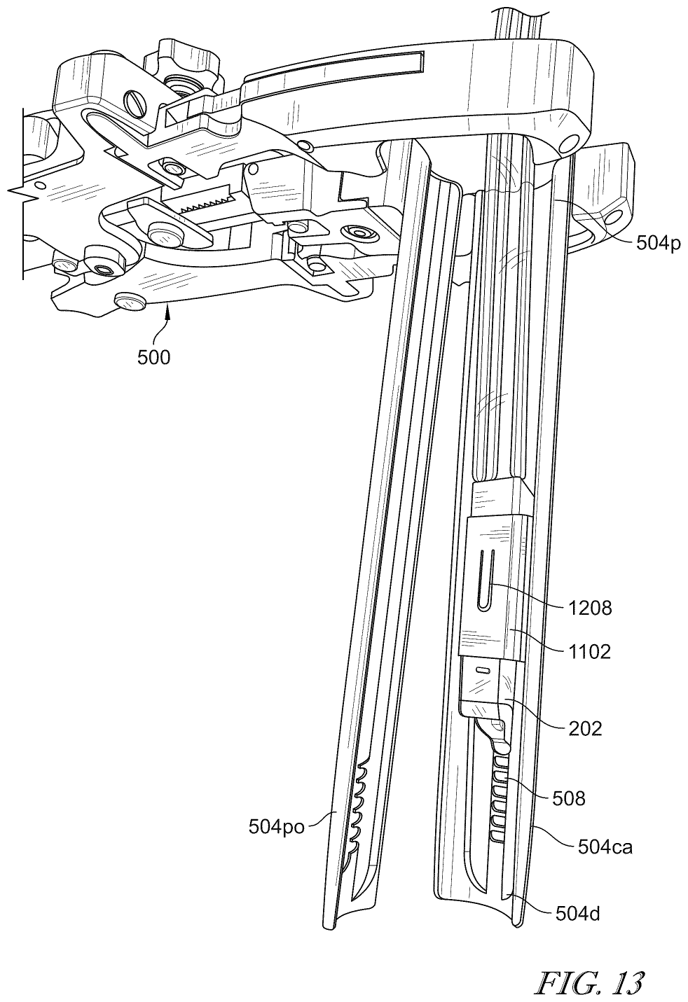

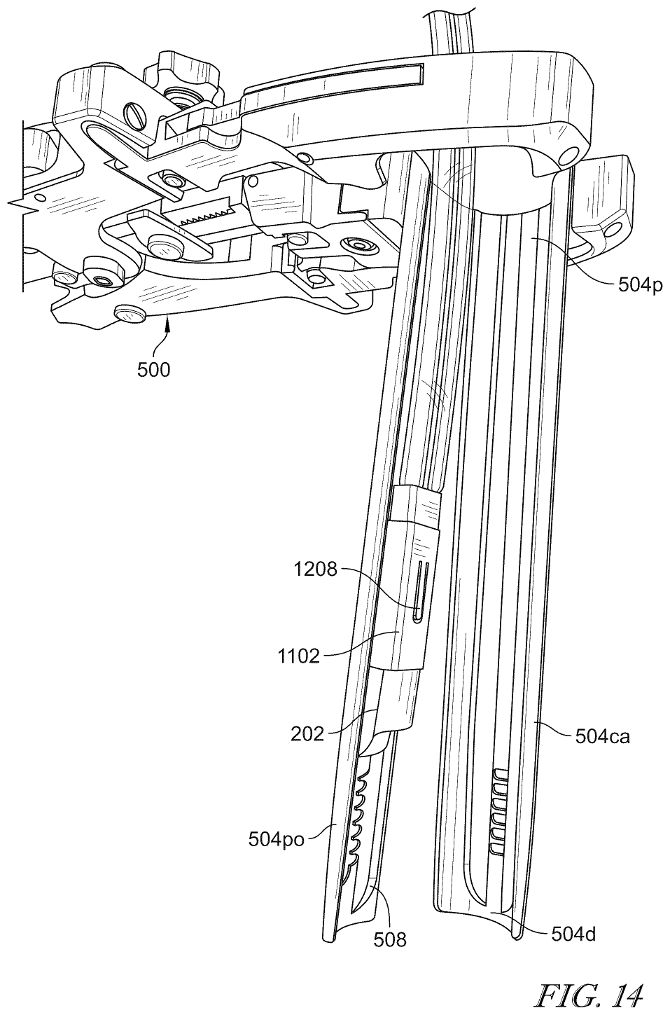

In some embodiments, the camera 202 can be used in an LLIF or other procedure using an expanding blade retractor, e.g., like the retractor 500 in FIG. 5, and can be mounted to either the posterior 504po, cranial 504cr, or caudal 504ca retractor blades. For example, FIG. 13 illustrates the retractor 500 with the camera 202 coupled to the caudal retractor blade 504ca while FIG. 14 illustrates the retractor 500 with the camera 202 coupled to the posterior retractor blade 504po. In both of these figures, the cranial retractor blade 504cr is removed to provide visibility. While this is also possible in use, in some embodiments the cranial retractor blade 504cr can be present, as shown in FIG. 8, for example. Further, the camera 202 can also be coupled to the cranial retractor blade 504cr in a similar manner as the other two retractor blades 504ca, 504po. All three of the retractor blades 504 can have a similar slot 508 formed along a surface thereof along their length and can interface with the camera 202 in a similar manner using the camera adapter 1102.

In some embodiments, a surgeon or other user may prefer to mount the camera 202 along a length of either the posterior retractor blade 504po, e.g., as shown in FIG. 14, or an anterior fourth retractor blade 1104, e.g., as shown in FIG. 8. In certain embodiments, one of these two positions can offer the best access for the procedure, though in other embodiments either of the cranial or caudal retractor blades 504cr, 504ca can be utilized to mount the camera 202.

While the retractor blades disclosed herein have been labeled with various anatomical direction terms (e.g., cranial, caudal, posterior, anterior) based on the arrangement of a patient and the retractor 600 shown and discussed in FIGS. 27-31 below, in certain embodiments the retractor can be positioned in a different manner and the relevant anatomical direction terms can change for each component. For example, if the retractor were rotated 180 degrees about a longitudinal axis of the working channel 610 from the position shown in FIG. 27 the cranial/caudal labels would reverse for the blades 604ca, 604cr, and the anterior/posterior labels would reverse for the blades 604po and 1104. Alternatively, if the patient were flipped about their cranial/caudal axis from the position shown in FIG. 27 to approach from the opposite lateral side, the anterior/posterior labels would reverse for the blades 604po and 1104 while the cranial/caudal labels for the blades 604ca, 604cr might remain the same (e.g., if the retractor 600 was not moved relative to its position in FIG. 27). Use of the components disclosed herein in any of a variety of positions relative to the patient is considered within scope of the present disclosure.

Returning to the fourth retractor blade 1104, it can be desirable to provide an instrument to allow for manual manipulation of the fourth retractor blade and for selective fixation thereof in a desired position. The blade holder 1106 provides such functionality and FIGS. 13-22 provide detail views of certain embodiments of the blade holder. The blade holder 1106 can be formed in different sizes and configurations to accommodate different retractor blades. For example, a version of the blade holder 1106 can be made for both the narrower posterior/anterior retractor blades and the wider cranial/caudal blades. The blade holder 1106 can allow the retractor blade to be used as a free-hand retractor blade (e.g., similar to a Love retractor), and can accept any length of blade.

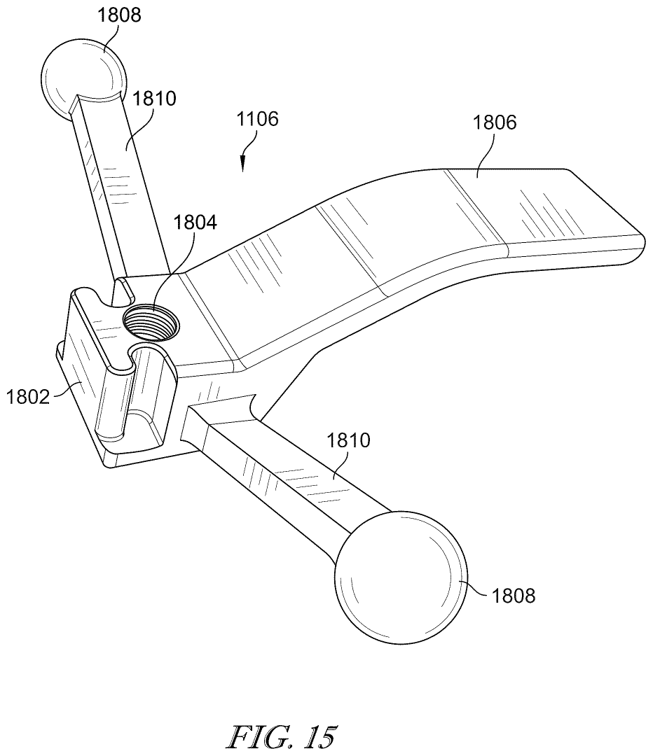

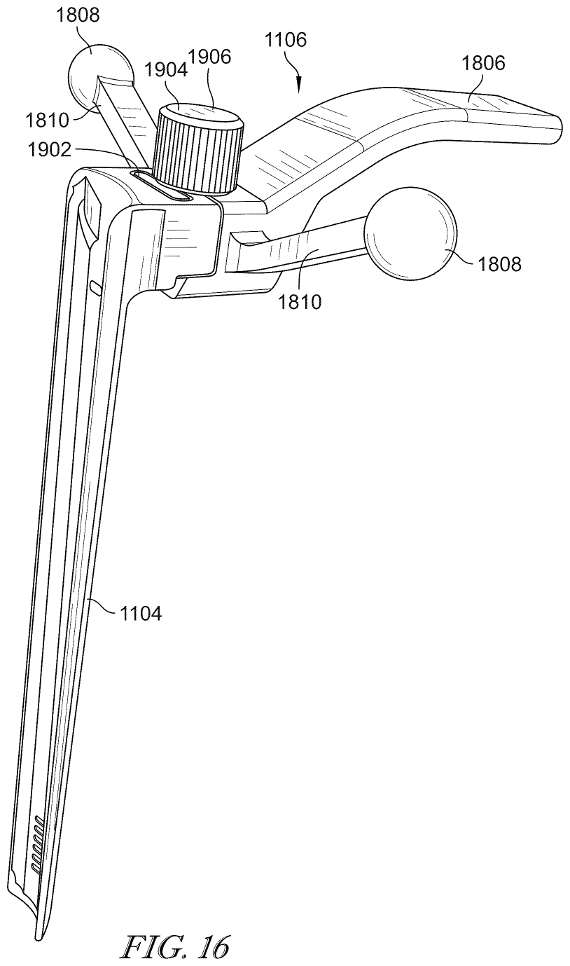

As shown in FIG. 15, the blade holder 1106 can include a male dovetail feature 1802 configured to interface with a female dovetail feature 1902 formed at a proximal end of the retractor blade 1104. In some embodiments, such as an embodiment where the blade holder 1106 is configured to couple with one of the cranial/caudal blades 504cr, 504ca, the reverse configuration can be employed with a female dovetail feature on the blade holder configured to couple with a male dovetail feature formed on the retractor blade. The blade holder 1106 can also include a threaded bore 1804 or other feature configured to assist in retaining the retractor blade relative to the blade holder 1106. In the illustrated embodiment, for example, the threaded bore 1804 can receive a retention bolt 1904 therein, as shown in FIG. 16. The retention bolt 1904 can include a threaded distal portion configured to extend into the bore 1804 and an enlarged proximal portion 1906 that can contact a proximal surface of the retractor blade 1104 and prevent its movement proximally relative to the blade holder 1106, thereby preventing unintended separation of the blade holder and retractor blade.

The blade holder 1106 can also include a handle 1806 extending away from the dovetail coupling feature 1802. The handle 1806 can facilitate a user grasping the assembly of the blade holder 1106 and the retractor blade 1104 in order to adjust its position.

The blade holder 1106 can also include a pair of spheres 1808 on extension arms 1810, which can allow the blade holder (and blade coupled thereto) to mate with the connector arm 104 or other component adapted to utilize a ball-and-socket coupling (e.g., a table mount adapter, etc.). Using these features, a surgeon or other user can position the retractor blade 1104 where desired and then use a table-mounted retractor arm 902, the connector arm 104, or both, to lock the retractor blade in place without needing to hand-hold the retractor at all times. When using the connector arm 104 with its single actuation locking/unlocking mechanism 108 (e.g., actuating a single lever or element can lock or unlock both ends of the connector arm and its central pivoting joint), in-situ loosening, re-positioning, and re-tightening can be provided in a similar easy manner as the system 100 described above.

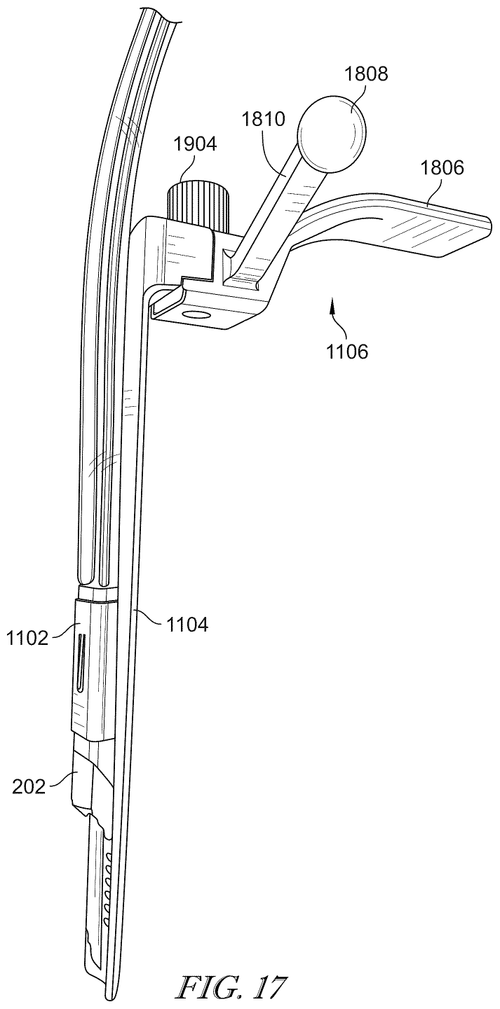

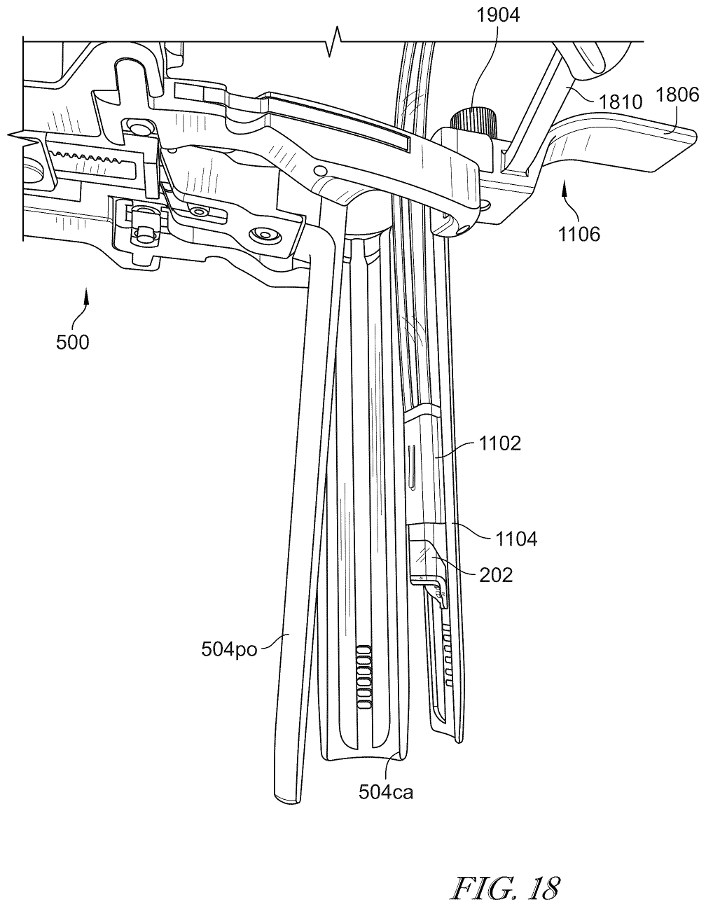

FIGS. 16 and 17 illustrate the retractor blade 1104 coupled to the blade holder 1106. FIG. 17 illustrates the camera 202 coupled to the retractor blade 1104 via the camera adapter 1102. FIG. 18 illustrates one example position of the retractor blade 1104 with camera 202 mounted thereto relative to the retractor 500 and its caudal blade 504ca and posterior blade 504po (the cranial blade 504cr is removed to provide visibility).

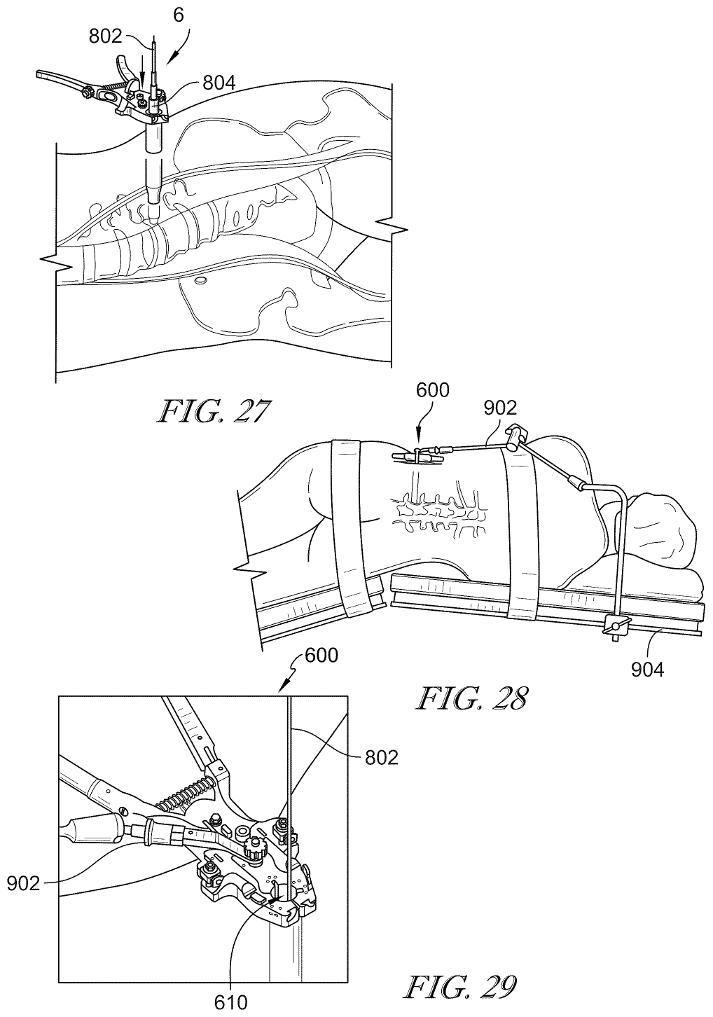

As noted above, when coupled with the camera 202 and camera adapter 1102, the retractor blade 1104 can also serve as the mount for the camera, allowing the surgeon to utilize the camera for better visualization in any procedure, regardless of approach or other retraction methods. That is, the retractor blade 1104 can be mounted independently of the retractor using a table mount arm (e.g., arm 902 shown in FIG. 28) or a connector arm (e.g., arm 104 shown in FIG. 1) mounted either to an implanted bone anchor (as shown in FIG. 1) or to a retractor (as shown in FIG. 8). Further, use of the blade holder 1106 and camera adapter 1102 can allow the retractor blade 1104 to replace the access port 102 in the system 100 shown in FIG. 1. The versatility of these components can allow for use in connection with any surgical approach to the spine, i.e., not solely a lateral approach and not solely with regard to a procedure in the lumbar region of the spine. These components can have application at any approach trajectory and along any region of the spine, among other potential surgical applications.

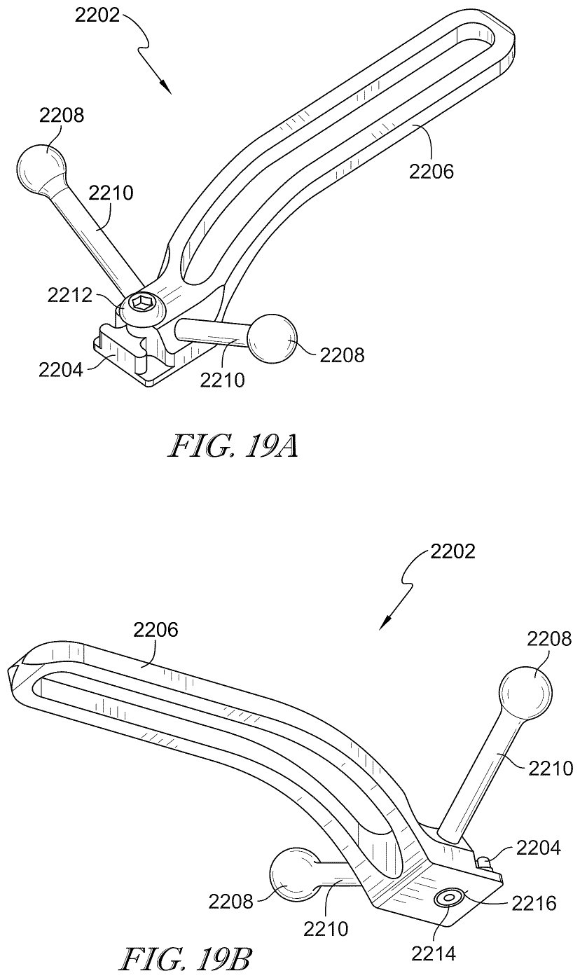

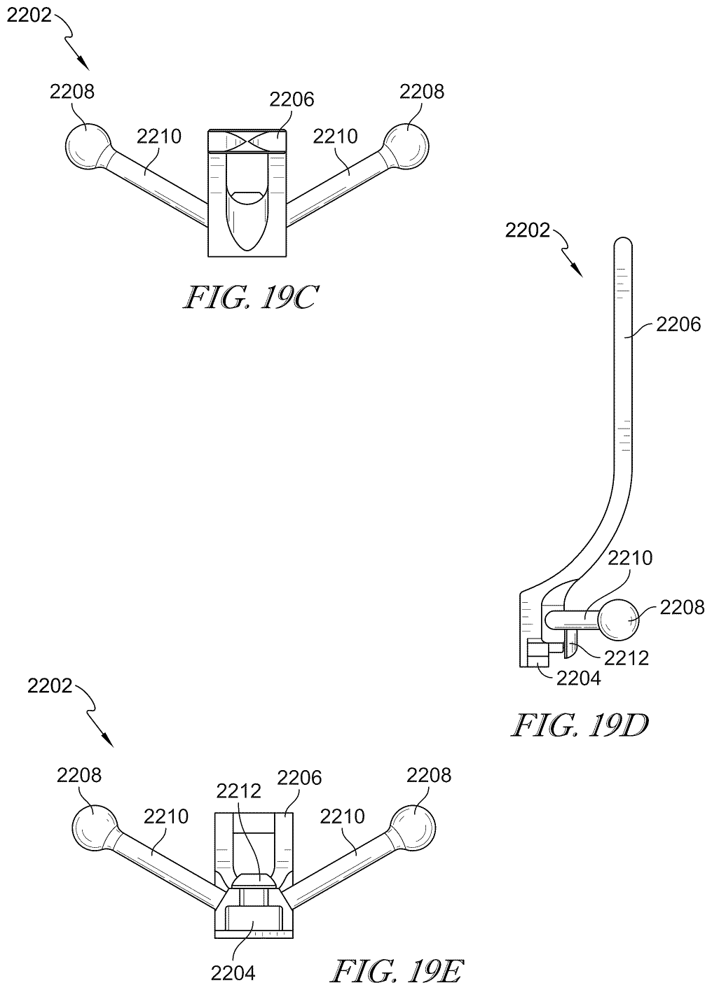

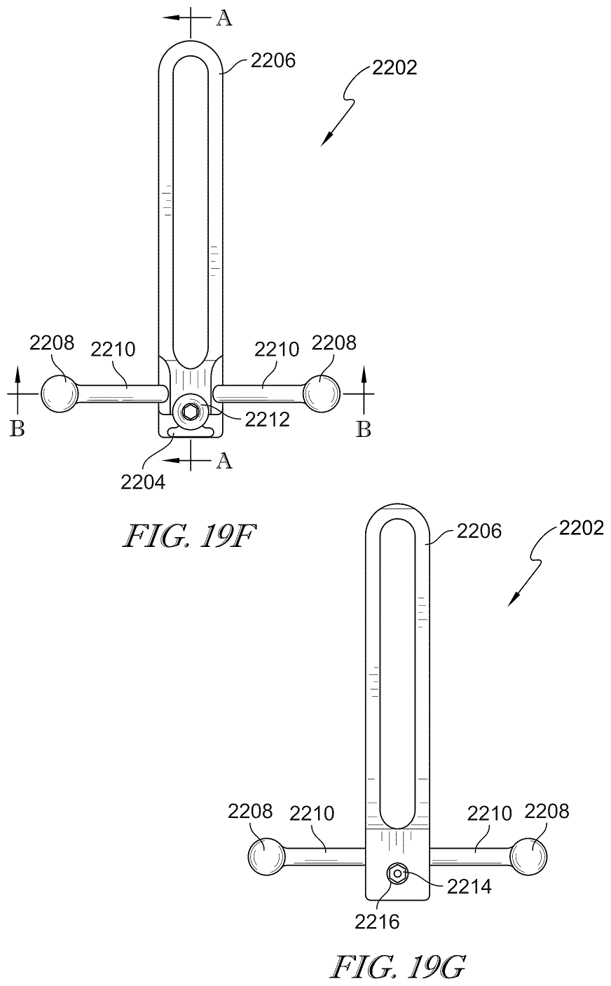

FIGS. 19A-22 illustrate another embodiment of a blade holder 2202. The blade holder 2202 operates similarly to the blade holder 1106 and repeated detailed description thereof will be omitted for sake of brevity. Generally speaking, the blade holder 2202 includes a similar dovetail mating feature 2204 to couple with the retractor blade 1104, a handle 2206 extending away from the dovetail mating feature to facilitate hand-holding and manipulation of the retractor blade 1104, and a pair of spheres 2208 on extension arms 2210 that are disposed on opposite sides of the handle 2206.

The form of the handle 2206 of the blade holder 2202 is different from the blade holder 1106, i.e., having the form of an elongated ring with a central opening. The alternative structure can have advantages associated with less material cost and weight, but the function is similar to the handle 1806 of the blade holder 1106.

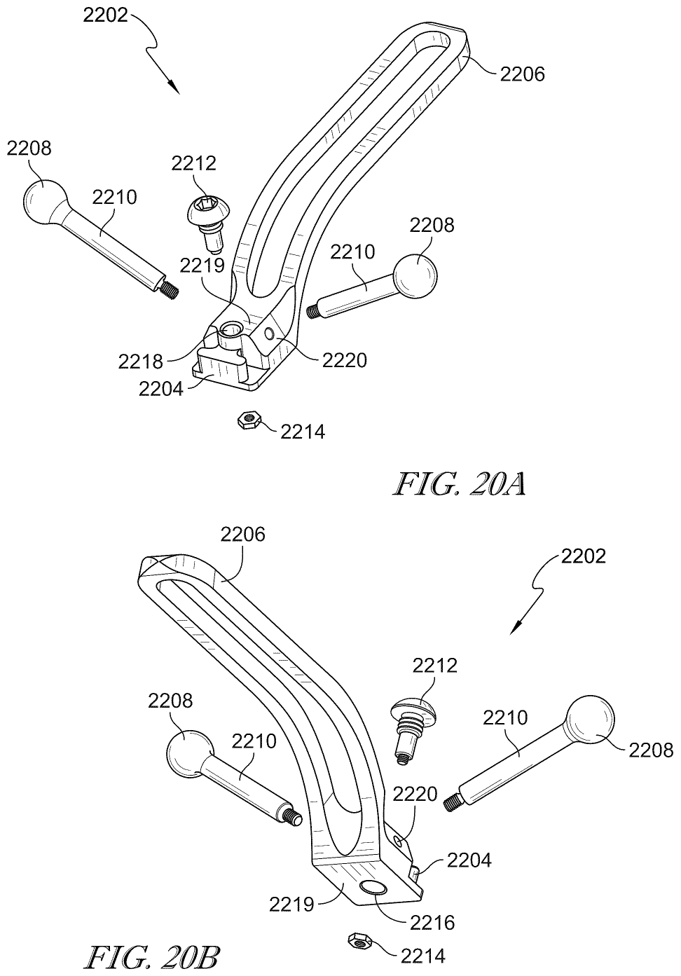

The blade holder 2202 can also include an alternative retention bolt 2212. The retention bolt 2212 can have a retention nut 2214 threaded to a distal portion thereof. The bolt 2214 can be received within a bore 2216 formed in the blade holder 2202 opposite the head of the bolt 2212. A proximal portion of the bolt 2212 extending distally from the head thereof can have threads formed thereon that are configured to interface with threads formed on a first portion 2218 of the bore 2216. This threaded interaction can provide the same locking of the retractor blade 1104 to the blade holder 2202 as the bolt 1904 did with the blade holder 1106. The bolt 2212 can also include a non-threaded portion extending distally from the proximal threaded portion, as well as a distal-most portion with threads formed thereon that are configured to interface with the threads of the nut 2214. Once the bolt 2212 is disposed within the bore 2216 and the nut 2214 threadably coupled to a distal end of the bolt, the bolt can be captured relative to the blade holder 2202 such that it cannot be separated therefrom. In comparison, the bolt 1904 can be separated from the blade holder 1106 as soon as its threads are disengaged from the bore 1804. Capturing the bolt 2212 relative to the blade holder 2202 can prevent unintended separation of the bolt from the blade holder while still allowing for selective locking of the retractor blade 1104 to the blade holder by engaging the proximal threaded portion of the bolt with the threads of the first portion 2218 of the bore 2216. The exploded views of FIGS. 20A and 20B, as well as the longitudinal cross-sectional view of FIG. 21, help illustrate this configuration.

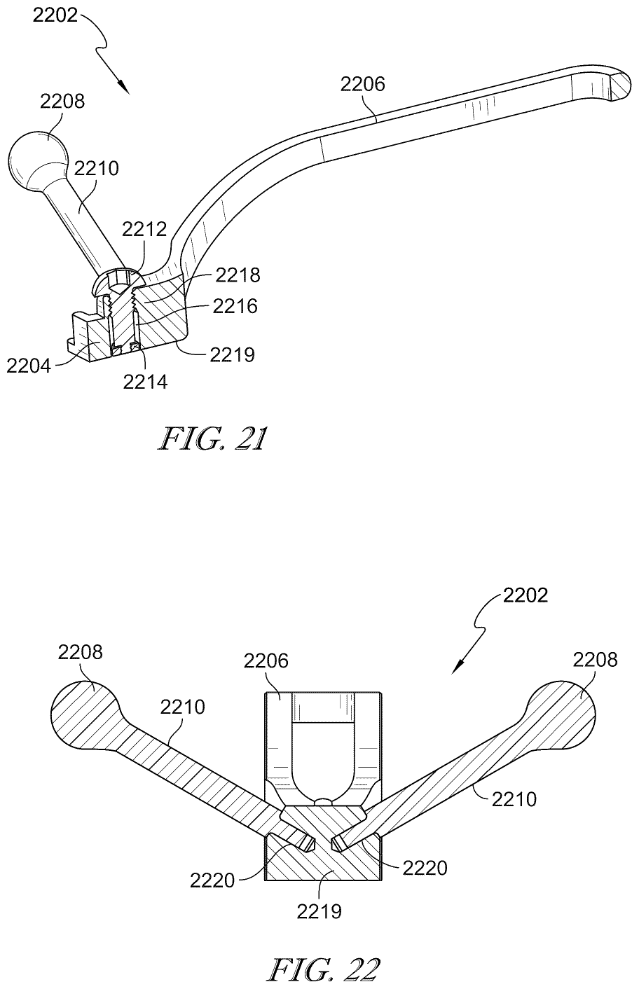

The exploded views of FIGS. 20A and 20B, as well as the lateral cross-sectional view of FIG. 22, illustrate that the spheres 2208 and extension arms 2210 are coupled to a body 2219 of the blade holder 2202 via a threaded connection (as opposed to being integrally formed in the embodiment of FIG. 15). In particular, a body portion 2219 of the blade holder 2202 can include opposed threaded bores 2220 formed therein that can each receive a threaded terminal end of one of the extension arms 2210. This modular configuration can provide advantages associated with manufacturing efficiency/cost and can allow any unused extension arm and sphere to be removed if desirable to avoid interference with other surgical instrumentation, users, etc.

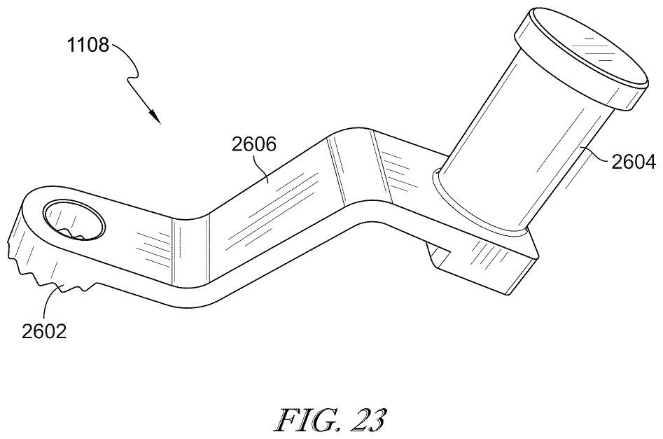

FIG. 23 illustrates the connector arm adapter 1108 in greater detail. The connector arm adapter 1108 can couple to an unused mounting point 512 on the retractor 500 (the other mounting point 512 can be utilized, e.g., to mount the retractor relative to the operating table or other anchoring structure), thereby allowing the connector arm 104 to be mounted to the retractor. When combined with the camera adapter 1102 and blade holder 1106, the connector adapter 1108 can allow for an extra retractor blade 1104 to be used as a fourth blade (e.g., an anterior blade). Because of the configuration of the various components disclosed herein, this fourth blade can have better adjustment and locking than the fourth blade mounting options that are often provided with retractors like those shown in FIGS. 5 and 6 (e.g., a simple crossbar extending between the ends of the opposed arms carrying the cranial/caudal retractor blades). Further, use of the components disclosed herein can allow the fourth retractor blade 1104 to carry the camera 202 for increased visualization of any procedure.

As shown in FIGS. 8 and 23, the connector arm adapter 1108 can include a mount 2602 configured to interface with any of the mounting points 512 on the retractor, e.g., a castellated or multi-tooth locking feature in the illustrated embodiment. The connector arm adapter 1108 can be coupled to a mounting point 512 of the retractor 500 using a bolt 1110 driven through a bore formed in the mount 2602 of the adapter. The connector arm adapter 1108 can also include a post 2604 offset from the mount 2602 by an arm 2606. The post 2604 can be used to provide a mounting cylinder for the connector arm 104 to rasp and couple to the retractor 500. The post 2604 can have a diameter configured to interface with one end of the connector arm 104, e.g., a diameter similar to the anchor 106 shown in FIG. 1 to which one end of the connector arm 104 is coupled. The arm 2606 can have any of a variety of shapes, including shapes that provide various degrees of offset in different axes in order to avoid interference with operation of the retractor, surgical workflow, etc.

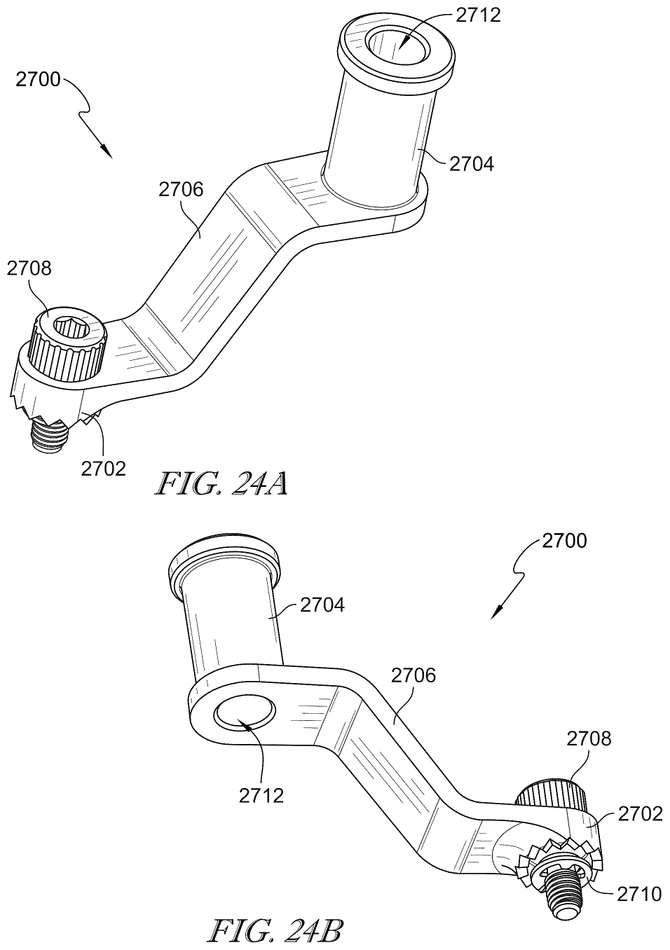

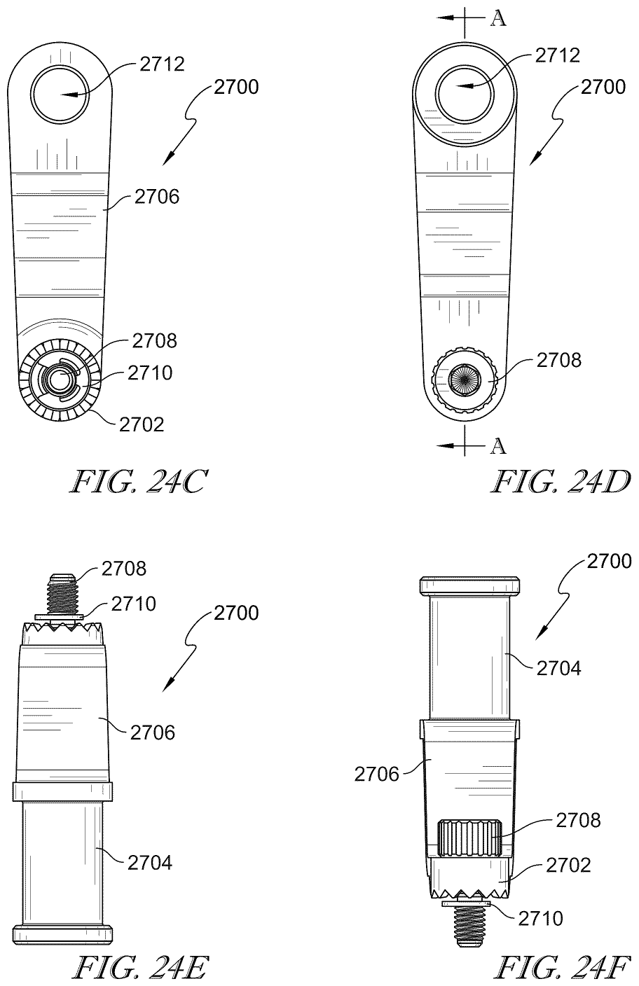

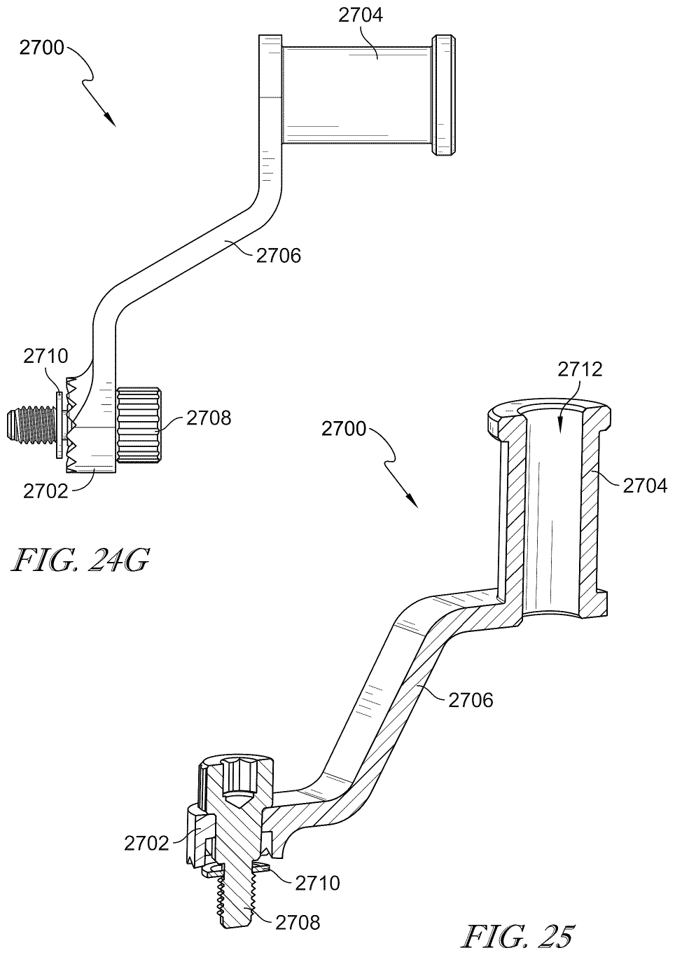

FIGS. 24A-26B illustrate another embodiment of a connector arm adapter 2700. The connector arm adapter 2700 operates similarly to the connector arm adapter 1108 and repeated detailed description thereof will be omitted for sake of brevity. Generally speaking, the connector arm adapter 2700 includes a similar mount 2702 to couple with the retractor 500, a post 2704 for coupling with the connector arm 104, and an arm 2706 that offsets the post 2704 from the mount 2702 in one or more axes.

Similar to the blade holder 2202, the connector arm adapter 2700 includes a captured bolt 2708 extending through a bore formed in the mount 2702 that can be utilized to secure the adapter relative to the retractor 500. In the embodiment of the connector arm adapter 2700, a spring clip 2710 is disposed around the bolt 2708 at a position proximal to its threaded distal-most portion but on an opposite side of the mount 2702 from the head of the bolt. This can allow some degree of relative translation of the bolt along its longitudinal axis relative to the connector arm adapter 2700, but prevents unintended separation of the bolt from the adapter.

Another difference between the connector arm adapter 1108 and the adapter 2700 is the presence of a through bore 2712 extending through the post 2704. This can reduce component weight and cost, but the post functions similarly in each embodiment.

Each of the various components disclosed herein provides additional utility and versatility to improve visualization in any of a number of surgical procedures. For example, using the camera adapter 1102 can allow the camera 202 to be mounted to any of the retractor blades of a given expanding blade retractor. Further, the addition of two more components can allow a retractor blade to be used as a fourth blade during a lateral access procedure and/or as a table-mounted retractor blade with camera integration. Using the blade holder 1106, a posterior blade (of any length) can be mounted thereon and used as a hand-held retractor and/or mounted to the connector arm 104 or a table mounted arm 902 using the sphere connection points of the blade holder. Other connector types (such as the fork or Hudson connections, etc.) can also be used and are considered within the scope of the present disclosure. In such a configuration, the connector arm 104 can be utilized to lock and hold a fourth retractor blade, allowing for a rigid and infinitely adjustable fourth blade mount (compared to the existing fourth blade retractor mount options, which are not rigidly locked). When combined with the camera adapter 1102, the camera 202 can also be mounted to this additional blade and used as a table mounted, retractor mounted, or handheld base for the camera 202. In such a configuration, the camera 202 can allow for visualization of the lateral disc space from either the anterior or posterior side of the surgical site, e.g., annulotomy.

FIGS. 27-31 illustrate use of the example access system including the retractor of FIG. 6 in a minimally invasive lateral interbody fusion using a lateral, retroperitoneal approach. The disclosed access systems can be used in various spinal surgery approaches, including lateral approaches in which a working corridor is established through a patient's tissue from a lateral skin surface to the spine. In these procedures, the patient can be positioned in a lateral decubitus position (i.e., laying on a side) and a working corridor can be created from a lateral side of the patient perpendicular to the ground, as shown in FIGS. 27-29. In some embodiments, the patient can be positioned in a prone position (i.e. laying on stomach) and the working corridor can be created from a lateral side of the patient parallel to the ground. In the lateral decubitus position, the patient may need to be flipped into a prone position for certain steps, including spinal rod and screw insertion that can require access to the posterior portion of the spine. A prone position can therefore be advantageous to avoid patient movement during the procedure, but the lateral approach that is parallel to the ground when the patient is lying prone can require a surgeon or other user to crouch down or lean over to directly visualize the surgical site through the working channel (as opposed to, for example, looking down into the working channel that extends perpendicular to the floor when the patient is on their side). The camera and access systems disclosed herein can provide an ergonomic benefit to a surgeon or other user by providing visualization in a manner that avoids the need for the surgeon to hunch or bend down to directly visualize the surgical site through the working channel that is extending parallel to the ground in the prone position. As a more specific example, mounting the camera 202 to the fourth retractor blade 1104 in the anterior position can be beneficial for ergonomic visualization when the patient is in the prone position. Further, the camera systems disclosed herein can be mounted to various radial positions about a perimeter of the working channel defined by the access device. The visualization systems disclosed herein can be used in conjunction with neuromonitoring and fluoroscopy to identify the target surgical site and nearby nervous system structures. This said, an advantage of utilizing the visualization systems and methods disclosed herein is an ability to eliminate or minimize use of fluoroscopy or other radiological imaging techniques that can have undesirable side effects, such as exposure of the patient to radiation.

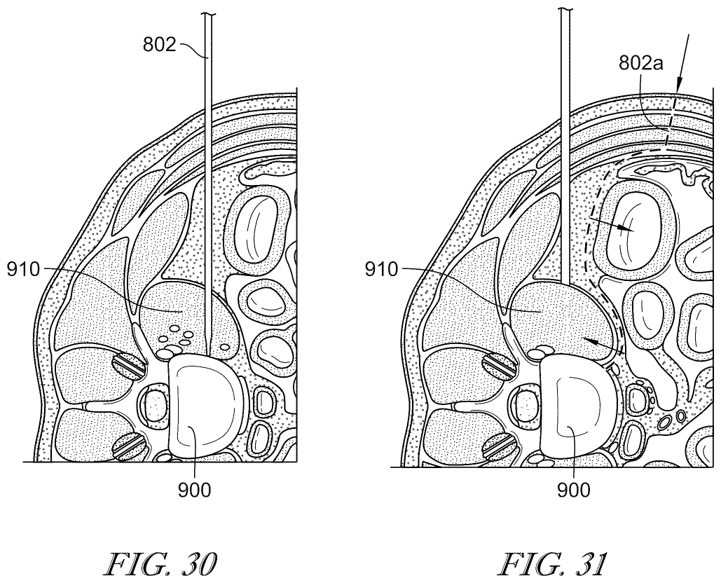

In the illustrated procedure, the retractor 600 can be positioned to provide a working corridor to a surgical site from a skin surface of a patient, as shown in FIG. 30. The retractor 600 can extend through the psoas muscle, as shown in FIG. 30, and hold the working corridor open to facilitate the procedure. After insertion of an initial probe 802 through the psoas muscle 910 and subsequent introduction of sequential dilators 804 (see FIG. 27), the retractor 600 can be positioned over the largest dilator and secured using an arm 902 coupled to the operating table 904 or other anchoring structure, as shown in FIG. 28. The sequential dilators 804 can be removed and the working corridor 610 utilized for the procedure.

The adjustable visualization provided by the embodiments disclosed herein can allow a surgeon to perform a lateral procedure without—or at least with reduced—use of radiological visualization. For example, the distal end of the retractor blades can be docked at various depths and/or the camera 202 can be positioned at various points along the length of the accessory slots 508 of the retractor blades while dissecting soft tissue to reach a surgical site, such as the patient's spine. In some embodiments, it can be beneficial to dock the retractor blades or position the camera 202 in a shallow position to better visualize and/or avoid the psoas muscle or other anatomic structures near the surgical site. In some embodiments, after the working corridor is established, the distal end of one or more of the retractor blades can be docked to a fixed position relative to the surgical site using anchors implanted in bone, such as bone pins, shims, or other fixation devices. During docking, the camera 202 and adaptor 1102 can be slid along the slot 508 to visualize the docking of the distal end of the retractor blades. The camera or visualization systems disclosed herein can provide advantages over prior surgical procedures utilizing this approach because they can enable direct visualization of docking or anchoring the retractor or surgical access device to an anatomical feature at the surgical site, such as a vertebra. In prior procedures, such visualization was not possible or was limited, such as using periodic fluoroscopy images, to determine the position of the access device, spine, and any pin, shim, or other fixation device being utilized to anchor the surgical access device to the patient.

In some procedures, the working corridor can be established anterior the psoas muscle 910, as shown in FIG. 31, which can require traversing around the abdominal cavity and the psoas muscle 910 to reach the surgical site 900. In these procedures, a handheld retractor assembly, such as the assembly shown in FIGS. 8 and 16 including a fourth blade 1104, blade holder 1106, and camera system 202, can be used to traverse and visualize the soft tissue prior to inserting the retractor assembly. After this manual tissue dissection, the handheld retractor assembly can be coupled to the retractor body 602 of the access system via a connector arm adaptor 1108, as discussed with respect to FIGS. 8 and 23. The access system can be used to further retract soft tissue and establish the working corridor to the surgical site 900.

In either case, the camera system can be moved from one retractor blade to another to adjust the direction of the camera to visualize procedure steps, such as anchor insertion, dissection of muscle or soft tissue, annulotomy, discectomy, endplate preparation, trialing, graft insertion, or cage insertion, among other steps. For example, in some of these steps, the surgeon can use depth etchings or other markings on the tools as guidance. These can be difficult to see using prior techniques, but the field of view and adjustable positioning of the camera system 202 can allow the surgeon to directly and more clearly visualize these markers for better guidance during each step. Further, the camera system 202 can be removed from the working channel completely for certain steps in which direct visualization through the working channel is adequate. In the illustrated embodiment, the handheld retractor assembly can be coupled to the access system in an anterior position, opposing the proximal retractor blade 604po. In procedures performed anterior to the psoas muscle 910, mounting the camera system to either the proximal retractor blade 604po or fourth blade 1104 in the anterior position can be beneficial to view and avoid the anterior abdominal cavity or posterior psoas muscle during the procedure.

Example surgical methods for vertebral body fusion are described below. These procedures can be performed using a plurality of approaches, including a direct lateral approach (i.e., dissecting through the psoas muscle that runs along the side of the spine) with a patient in the lateral decubitus position (i.e., on their side), a direct lateral approach with a patient in a prone position (i.e., on their stomach), an anterior to psoas approach (i.e., approaching the spine from a position anterior to the psoas muscle to avoid dissecting through the psoas muscle).

An example surgical method performed using a direct lateral approach with a patient in the lateral decubitus position (i.e., on their side) or a prone position (i.e., on their stomach) can include forming an incision, dilating the incision to a desired size using, e.g., sequential dilators, and inserting a surgical access device or system, such as a multi-bladed expandable surgical retractor described herein (the dilators can also be removed once the access device is inserted). The multi-bladed expandable surgical retractor can be advantageous in lateral approach procedures relative to, for example, a fixed-size tubular or other access port because lateral approaches can be deeper compared to other approaches and can involve intervertebral cages or implants that are larger than required with other procedures (e.g., procedures utilized higher in the spine where vertebrae are smaller). In addition, it can be desirable to minimize the time and degree of retraction performed on muscle surrounding the spine, especially the psoas, due to concerns for causing damage to the muscle and/or nerves extending therethrough/adjacent thereto. In such cases, multi-bladed expandable retractors can offer advantages because they can be inserted in a smaller configuration and expanded only as much as needed for as long as needed during a procedure. In addition, many expandable bladed retractors include the ability to toe the blades relative to one another (i.e., angle distal ends of the blades away from one another while maintaining a distance between proximal ends of the blades). This can allow greater retraction at a surgical site while minimizing retraction of muscle or tissue located more remotely from the surgical site. This said, fixed size access ports can be utilized in connection with the visualization systems and methods disclosed herein as well.

The method can further include mounting the surgical access device to a surgical table or bed upon which the patient is positioned. The method can also include inserting or mounting a visualization system as described herein to the surgical access device, e.g., by mounting a camera sensor to a distal portion of the access device using any of the adapters described herein such that the camera sensor can be positioned within the patient's body adjacent to a surgical site. Utilizing a camera sensor or visualization system that places a camera sensor in the body at the surgical site can provide direct visualization to a user without requiring the user to manipulate controls of a more traditional endoscope, etc. This can provide hands-free direct visualization of a surgical site throughout a procedure.

In some embodiments, the camera or visualization system can be utilized to monitor docking of a distal portion of the surgical access device to the patient's anatomy, e.g., the spine. For example, the camera can be utilized to observe placement of a vertebral body bone pin, disc shim, or other anchoring device in a patient's vertebra. In some embodiments, it can be desirable to position the camera such that it views the surgical site from the cranial or caudal perspective to best observe this step of the procedure.

Once docked to the patient's anatomy, the retractor can be expanded as needed to provide the desired working channel to the surgical site. If the patient is in the lateral decubitus position, the working channel can be oriented perpendicular to the floor and a surgeon or other user may be able to easily directly visualize the surgical site by looking down through the working channel. If the patient is in the prone position, the working channel can be oriented parallel to the floor and the camera or visualization system can provide a significant ergonomic benefit because it can provide visualization through the working channel without requiring the surgeon or other user to bend over in order to directly view through the working channel.

In some embodiments, the method can include coupling a 4^{th} retractor blade to a three-bladed expandable retractor. The method can also include adjusting a position of the camera or visualization system from the previously-noted cranial/caudal perspective to an anterior or posterior perspective (i.e., parallel to the disc space between adjacent vertebra). Under either direct or camera visualization (depending on patient position and surgeon or other user preference), dissection of any additional muscle or tissue can be performed to expose the desired surgical site. In some cases, initial insertion of the retractor may be intentionally done to a shallow depth short of the desired surgical site to avoid unintentionally damaging any nerves or other structures near the patient's spine. In such cases of “shallow docking,” the visualization system can allow a surgeon or other user to carefully dissect the remaining muscle and/or tissue while monitoring progress visually until the desired surgical site is exposed.

The method can further include performing any of a variety of surgical procedures at the surgical site. These can include annulotomy, discectomy, and vertebral endplate preparation, among others. These procedures can be performed using the visualization system if desired or the visualization system can be selectively removed to free up space in the working channel. Further, the visualization system can be utilized to assess progress and results of any of these operations. For example, the camera sensor of the visualization system can be inserted into the intervertebral disc space to observe the results of these procedures and assess readiness for subsequent steps. The field of view of the camera sensor can facilitate visualization of both vertebral endplate walls when oriented in the anterior/posterior direction (i.e., parallel with the disc space).

The method can further include returning the visualization system to a position where it is docked to a retractor blade and viewing the disc space from the anterior/posterior direction (i.e., parallel with the disc space), and utilizing it to observe insertion of one or more trialing instruments to determine an appropriate implant size. Trialing instruments typically have depth or other etchings formed thereon that can be difficult to read when inserted into an intervertebral disc space, but the visualization system can provide a close and direct view that can make this easier.

The method can further include inserting bone graft material into the intervertebral space and observing insertion using the visualization system. The close and direct view from the camera can allow better determination of proper bone graft amount and placement.

The method can also include inserting an intervertebral implant, such as an intervertebral fusion cage, into the intervertebral space and observing insertion using the visualization system. Again, the close and direct view from the camera can allow better placement of the implant and facilitate determinations regarding potential need for additional graft material, repositioning, etc.

Finishing up the surgical procedure can include collapsing the retractor or surgical access device, removing any bone pin, disc shim, or other anchoring device used to dock the access device to the spine, disconnecting the retractor or access device from the operating table (or arm coupled thereto), and removing the access device from the patient. The visualization system can remain coupled to the retractor or access device during this process and, as a result, a surgeon or other user can observe muscle and/or tissue as the access device is removed from the patient to avoid any unintended damage thereto and/or monitor for any potential issue.

As noted above, the systems and methods disclosed herein can be applied to a variety of surgical procedures. Another example procedure is an anterior to psoas lateral approach to the spine. In this procedure, the spine is approached from a position anterior to the psoas muscle to avoid the need to dissect through the psoas (as in the direct lateral approaches described above). The approach is similar in many respects and, as a result, only certain differences will be addressed specifically. While this approach can avoid the need for dissecting through the psoas, it involves passing closer to the abdominal cavity and great vessels that are positioned anterior to the spine. In view of this, manual dissection to the spine is common. Accordingly, in this procedure, the visualization system or camera sensor can be mounted to a handheld retractor blade, as described above in connection with FIG. 17. An incision can be formed in the patient and a surgeon or other user can use the handheld retractor blade with visualization system to bluntly dissect muscle and/or tissue to reach the spine, as shown in the approach of FIG. 31 (i.e., path 802a). The surgeon or other user can utilize the handheld retractor with visualization system to visualize the space between muscles and watch for anterior organs, the great vessels that are positioned anterior to the spine, and/or the psoas muscles.

Once the spine is reached, the surgical method can proceed in the same manner as described above for the direct lateral approaches, including insertion of an expandable retractor or other access device, docking the access device to the patient and/or operating table, retracting tissue to create the desired working channel, visualizing any additional dissection that may be needed, performing any of an annulotomy, discectomy, and endplate preparation under visualization of the camera, inspecting the intervertebral space using the visualization system, trialing implant sizes, inserting bone graft, inserting intervertebral implant, and finishing the surgical procedure. All of these steps can be performed under visualization of the camera in a similar manner as described above.

Various devices and methods disclosed herein can be used in minimally invasive surgery and/or open surgery. While various devices and methods disclosed herein are generally described in the context of surgery on a human patient, the methods and devices disclosed herein can be used in any of a variety of surgical procedures with any human or animal subject, or in non-surgical procedures.

Various devices disclosed herein can be constructed from any of a variety of known materials. Example materials include those that are suitable for use in surgical applications, including metals such as stainless steel, titanium, titanium nitride, nickel, cobalt, chrome, cobalt-chromium, or alloys and combinations thereof, polymers such as PEEK, ceramics, carbon fiber, and so forth. The various components of the devices disclosed herein can be rigid or flexible. In addition, one or more of the components or devices disclosed herein can be formed as monolithic or unitary structures, e.g., formed from a single continuous material, or can be formed from separate components coupled together in a variety of manners that either facilitate or discourage subsequent separation. One or more components or portions of the device can be formed from a radiopaque material to facilitate visualization under fluoroscopy and other imaging techniques, or from a radiolucent material so as not to interfere with visualization of other structures. Example radiolucent materials include carbon fiber and high-strength polymers. Further, various methods of manufacturing can be utilized, including 3D printing or other additive manufacturing techniques, as well as more conventional manufacturing techniques, including molding, stamping, casting, machining, etc.

Various devices or components disclosed herein can be designed to be disposed of after a single use, or they can be designed to be used multiple times. In either case, however, various devices or components can be reconditioned for reuse after at least one use. Reconditioning can include any combination of the steps of disassembly, followed by cleaning or replacement of particular pieces, and subsequent reassembly. In particular, a device or component can be disassembled, and any number of the particular pieces or parts thereof can be selectively replaced or removed in any combination. Upon cleaning and/or replacement of particular parts, the device or component can be reassembled for subsequent use either at a reconditioning facility, or by a surgical team immediately prior to a surgical procedure. Reconditioning of a device or component can utilize a variety of techniques for disassembly, cleaning/replacement, and reassembly. Use of such techniques, and the resulting reconditioned device or component, are within the scope of the present disclosure.

Various devices or components described herein can be processed before use in a surgical procedure. For example, a new or used device or component can be obtained and, if necessary, cleaned. The device or component can be sterilized. In one sterilization technique, the device or component can be placed in a closed and sealed container, such as a plastic or TYVEK bag. The container and its contents can be placed in a field of radiation that can penetrate the container, such as gamma radiation, x-rays, or high-energy electrons. The radiation can kill bacteria on the device or component and in the container. The sterilized device or component can be stored in the sterile container. The sealed container can keep the device or component sterile until it is opened in the medical facility. Other forms of sterilization are also possible, including beta or other forms of radiation, ethylene oxide, steam, or a liquid bath (e.g., cold soak). Certain forms of sterilization may be better suited to use with different devices or components, or portions thereof, due to the materials utilized, the presence of electrical components, etc.

In this disclosure, articles “a” and “an” are used to refer to one or to more than one (i.e., at least one) of the grammatical object of the article. By way of example, “an element” means at least one element and can include more than one element. The term “about” is used to provide flexibility to a numerical range endpoint by providing that a given value may be “slightly above” or “slightly below” the endpoint without affecting the desired result. The use herein of the terms “including,” “comprising,” or “having,” and variations thereof, is meant to encompass the elements listed thereafter and equivalents thereof, as well as additional elements. As used herein, “and/or” refers to and encompasses any and all possible combinations of one or more of the associated listed items, as well as the lack of combinations where interpreted in the alternative (“or”). Further, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B,” “one or more of A and B,” and “A and/or B” are each intended to mean “A alone, B alone, or A and B together.” A similar interpretation is also intended for lists including three or more items. For example, the phrases “at least one of A, B, and C,” “one or more of A, B, and C,” and “A, B, and/or C” are each intended to mean “A alone, B alone, C alone, A and B together, A and C together, B and C together, or A and B and C together.” In addition, use of the term “based on,” is intended to mean, “based at least in part on,” such that an un-recited feature or element is also permissible.

To the extent that linear, circular, or other dimensions are used in the description of the disclosed devices and methods, such dimensions are not intended to limit the types of shapes that can be used in conjunction with such devices and methods. Equivalents to such dimensions can be determined for different geometric shapes, etc. Further, like-numbered components of the embodiments can generally have similar features. Still further, sizes and shapes of the devices, and the components thereof, can depend at least on the anatomy of the subject in which the devices will be used, the size and shape of objects with which the devices will be used, and the methods and procedures in which the devices will be used.

The figures provided herein are not necessarily to scale. Still further, to the extent arrows are used to describe a direction of movement, these arrows are illustrative and in no way limit the direction that the respective component can or should be moved. Other movements and directions may be possible to create the desired result in view of the present disclosure.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Further features and advantages based on the above-described embodiments are possible and within the scope of the present disclosure. Accordingly, the disclosure is not to be limited by what has been particularly shown and described. All publications and references cited herein are expressly incorporated herein by reference in their entirety, except for any definitions, subject matter disclaimers, or disavowals, and except to the extent that the incorporated material is inconsistent with the express disclosure herein, in which case the language in this disclosure controls.

Examples of the above-described embodiments can include the following:
1. A surgical method, comprising:
   inserting a surgical access device into a patient using a lateral approach;
   mounting a visualization system to a distal portion of the access device at a first position about a perimeter of a working channel defined by the access device;
   coupling the surgical access device to a vertebra of the patient's spine using the visualization system to assess coupling progress;
   mounting the visualization system at a second position about the perimeter of the working channel of the access device;
   dissecting tissue to access the patient's spine using the visualization system at the second position to assess dissection progress;
   preparing the patient's spine for vertebral body fusion;
   inserting the visualization system into space between adjacent vertebrae to observe results of preparing the patient's spine for vertebral body fusion.
2. The method of example 1, further comprising forming an incision in a patient's skin.
3. The method of any of examples 1 to 2, further comprising adjusting a size of the working channel of the surgical access device after insertion into the patient.
4. The method of any of examples 1 to 3, wherein inserting the surgical access device into the patient is performed while the patient is in a lateral decubitus position.
5. The method of any of examples 1 to 3, wherein inserting the surgical access device into the patient is performed while the patient is in a prone position.
6. The method of any of examples 1 to 5, wherein dissecting tissue to access the patient's spine includes approaching the spine anterior to the psoas muscle.
7. The method of any of examples 1 to 5, wherein dissecting tissue to access the patient's spine includes dissecting through the psoas muscle.
8. The method of any of examples 1 to 7, further comprising mounting the surgical access device to a surgical table upon which the patient is disposed.
9. The method of any of examples 1 to 8, further comprising mounting an additional retractor blade to the surgical access device after the surgical access device is inserted into the patient.
10. The method of any of examples 1 to 9, wherein the first position for mounting the visualization system is oriented to view the patient's spine from a cranial or caudal direction.
11. The method of any of examples 1 to 10, wherein the second position for mounting the visualization system is oriented to view the patient's spine from an anterior or posterior direction.
12. The method of any of examples 1 to 11, further comprising returning the visualization system to the second position after inserting the visualization system into the space between adjacent vertebrae.
13. The method of any of examples 1 to 12, further comprising inserting one or more implant trialing instruments into the space between adjacent vertebrae using the visualization system to assess required implant size.
14. The method of any of examples 1 to 13, further comprising inserting bone graft material using the visualization system to assess insertion progress.
15. The method of any of examples 1 to 14, further comprising inserting an intervertebral implant using the visualization system to assess implant positioning.
16. The method of any of examples 1 to 15, further comprising separating the surgical access device from the patient's spine using the visualization system to assess separation progress.
17. The method of any of examples 1 to 16, further comprising removing the surgical access device from the patient while using the visualization system to monitor muscle and/or tissue during removal.
18. The method of any of examples 1 to 17, wherein the visualization system includes a camera sensor disposed at a distal end thereof such that mounting the visualization system to the distal portion of the access device includes inserting the camera senor into the patient.
19. A surgical method, comprising:
   inserting a distal portion of an access device into a patient through an incision, the access device defining a working channel therethrough;
   mounting a camera system into the working channel of the access device at a first position around a perimeter of the working channel;
   coupling the access device to a support arm;
   docking the access device to an anatomical structure of the patient at a surgical site, wherein the camera system is configured to view the anatomical structure in the first position;
   repositioning the camera to a second position around the perimeter of the working channel;
   performing one or more surgical procedures at the surgical site.
20. The method of example 19, wherein a plurality of camera systems are mounted at a plurality of positions around the perimeter of the access device.
21. The method of any of examples 19 to 20, wherein the working channel extends medially from a lateral skin surface towards a spine of the patient.
22. The method of any of examples 19 to 21, wherein the working channel extends through a psoas muscle.
23. The method of example 22, wherein the patient is positioned in a lateral decubitus position and the working channel extends perpendicular to a ground surface.
24. The method of example 22, wherein the patient is positioned in a prone position and the working channel extends parallel to a ground surface.
25. The method of any of examples 19 to 24, wherein the surgical access device comprises:
   a retractor body including first arm, a second arm opposed to the first arm, and a third arm; and
   first, second, and third retractor blades respectively coupled to each arm and defining the working channel longitudinally therebetween.
26. The method of example 25, further comprising moving each retractor blade relative the other retractor blades to retract soft tissue.
27. The method of any of examples 25 to 26, wherein the camera system is mounted to the first retractor blade and views the surgical site from the caudal direction in the first position.
28. The method of any of examples 25 to 26, wherein the camera system is mounted to the second retractor blade and views the surgical site from the cranial direction in the second position.
29. The method of any of examples 25 to 26, wherein the camera system is mounted to the third retractor blade and views the surgical site from the posterior direction in the second position.
30. The method of any of examples 19 to 29, wherein the camera system comprises an adaptor configured to slidably couple the camera system to a retractor blade of the access device.
31. The method of example 30, further comprising adjusting the position of the camera system along a length of the retractor blade, wherein the retractor blade defines a portion of the working channel.
32. The method of example 31, wherein the camera system is positioned proximal to a distal end of the retractor blade and configured to visualize docking of the distal end of the access device.
33. The method of any of examples 19 to 32, wherein the surgical access device comprises a handheld retractor assembly configured to be selectively coupled to the retractor body, the handheld retractor assembly comprising a fourth retractor blade and a blade holder coupled to a proximal end of the fourth retractor blade.
34 The method of example 33, further comprising:
   mounting the camera system to a distal end of the handheld retractor assembly;
   inserting a distal end of the removable retractor blade into the incision through soft tissue towards the surgical site;
   retracting soft tissue with the handheld retractor assembly while visualizing the soft tissue through the camera system; and
   coupling the handheld retractor assembly to the access device.
35. The method of example 34, wherein the camera system views the surgical site from an anterior direction when mounted to the handheld retractor assembly.
36. The method of any of examples 34 to 35, wherein the working channel extends anterior to a psoas muscle.
37. The method of any of examples 34 to 36, wherein the handheld retractor assembly is coupled to the access system via a connector arm coupled to a proximal end of the retractor body and the blade holder.

What is claimed is:

1. A surgical method, comprising:
inserting a surgical access device into a patient using a lateral approach;
mounting a visualization system to a distal portion of the access device at a first position about a perimeter of a working channel defined by the access device;
coupling the surgical access device to a vertebra of the patient's spine using the visualization system to assess coupling progress;
mounting the visualization system at a second position about the perimeter of the working channel of the access device;
dissecting tissue to access the patient's spine using the visualization system at the second position to assess dissection progress;
preparing the patient's spine for vertebral body fusion;
inserting the visualization system into space between adjacent vertebrae to observe results of preparing the patient's spine for vertebral body fusion.

2. The method of claim 1, further comprising forming an incision in a patient's skin.

3. The method of claim 1, further comprising adjusting a size of the working channel of the surgical access device after insertion into the patient.

4. The method of claim 1, wherein inserting the surgical access device into the patient is performed while the patient is in a lateral decubitus position.

5. The method of claim 1, wherein inserting the surgical access device into the patient is performed while the patient is in a prone position.

6. The method of claim 1, wherein dissecting tissue to access the patient's spine includes approaching the spine anterior to the psoas muscle.

7. The method of claim 1, wherein dissecting tissue to access the patient's spine includes dissecting through the psoas muscle.

8. The method of claim 1, further comprising mounting the surgical access device to a surgical table upon which the patient is disposed.

9. The method of claim 1, further comprising mounting an additional retractor blade to the surgical access device after the surgical access device is inserted into the patient.

10. The method of claim 1, wherein the first position for mounting the visualization system is oriented to view the patient's spine from a cranial or caudal direction.

11. The method of claim 1, wherein the second position for mounting the visualization system is oriented to view the patient's spine from an anterior or posterior direction.

12. The method of claim 1, further comprising returning the visualization system to the second position after inserting the visualization system into the space between adjacent vertebrae.

13. The method of claim 1, further comprising inserting one or more implant trialing instruments into the space between adjacent vertebrae using the visualization system to assess required implant size.

14. The method of claim 1, further comprising inserting bone graft material using the visualization system to assess insertion progress.

15. The method of claim 1, further comprising inserting an intervertebral implant using the visualization system to assess implant positioning.

16. The method of claim 1, further comprising separating the surgical access device from the patient's spine using the visualization system to assess separation progress.

17. The method of claim 1, further comprising removing the surgical access device from the patient while using the visualization system to monitor muscle and/or tissue during removal.

18. The method of claim 1, wherein the visualization system includes a camera sensor disposed at a distal end thereof such that mounting the visualization system to the distal portion of the access device includes inserting the camera senor into the patient.
